(19) United States
(12) Patent Application Publication
(10) Pub. No.: US 2024/0131356 A1
(43) Pub. Date: Apr. 25, 2024

(54) COMBINATION OF INTRACELLULAR AND EXTRACELLULAR PHOTOSENSITIZERS FOR TREATMENT OF BACTERIAL INFECTION

(71) Applicant: Vanderbilt University, Nashville, TN (US)

(72) Inventor: Eric Skaar, Nashville, TN (US)

(21) Appl. No.: 18/272,919

(22) PCT Filed: Jan. 27, 2022
(86) PCT No.: PCT/US22/14086

§ 371 Date: Jul. 18, 2023

Related U.S. Application Data
(60) Provisional application No. 63/142,251, filed on Jan. 27, 2021.

(51) Int. Cl. A61N 5/06

(52) U.S. Cl. CPC A61N 5/0624; A61N 5/062; A61N 2005/0658

(57) ABSTRACT

A method of inducing death or inhibiting growth or reproduction of a Gram-positive bacterial cell involves contacting the bacterial cell with a coproporphyrinogen oxidase (CgoX) activator, thereby promoting accumulation of coproporphyrin III (CPIII) in the bacterial cell, where the CPIII serves as an intracellular photosensitizer; contacting the bacterial cell with a photosensitizer conjugated to an iron-regulated surface determinant (Isd) protein targeting moiety, which Isd proteins are expressed on the service of the bacterial cell such that the conjugated photosensitizer serves as an extracellular photo sensitizer; exposing the bacterial cell to light having a first excitation wavelength of the intracellular photo sensitizer; and exposing the bacterial cell to light having a second excitation wavelength of the extracellular photosensitizer.

### RELATED APPLICATIONS

This application claims priority from U.S. Provisional Application Ser. No. 63/142,251 filed Jan. 27, 2021, the entire disclosure of which is incorporated herein by this reference.

### GOVERNMENT INTEREST

This invention was made with government support under grant number RO1AI073843 awarded by the National Institutes of Health. The government has certain rights in the invention.

### TECHNICAL FIELD

The presently-disclosed subject matter generally relates an improved method of treating a bacterial infection, as an effective alternative to methods using antibiotic compounds. In particular, certain embodiments of the presently-disclosed subject matter make use of photodynamic therapy (PDT) involving methods designed to simultaneously expose Gram-positive bacteria to intracellular and extracellular photoactivators.

### INTRODUCTION

Skin and soft tissue infections (SSTIs) are responsible for more than 14 million ambulatory visits annually and many of these infections are caused by the Gram-positive bacterium Staphylococcus aureus (S. aureus) (1). This problem is exacerbated by the rise of antibiotic resistance in bacterial infections, limiting the number of effective treatment methods and creating an arms race between pharmacological development and bacterial evolution.

The Centers for Disease Control and Prevention lists methicillin-resistant S. aureus (MRSA) as Threat-level Serious and reported ˜323,700 cases of hospitalization, 10,600 deaths, and $1.7 billion of direct cost associated with MRSA infection in 2017 (2).

In addition to MRSA, methicillin-susceptible S. aureus infections are responsible for numerous cases of hospital-acquired and community-acquired bloodstream infections, which have a 24% and 14% mortality rate, respectively (3).

The rise of resistance to traditional antibiotic-based therapies in S. aureus and other Gram-positive pathogens highlights a critical need in the art for alternative therapies, which effectively treat infection, while impeding the evolution of therapeutic resistance.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter is directed to a treatment against Gram-positive pathogens, which serves as an alternative to traditional antibiotic treatment, and which, by design, impedes the evolution of antibiotic resistance in bacteria.

Heme is a key nutrient for S. aureus and is required for many important processes including electron transport, the transport of oxygen, and degradation of heme by heme oxygenases to acquire the nutrient iron (4-7). Heme biosynthesis is a promising area for the development of antimicrobial therapeutics due to the discovery of a divergent pathway for heme biosynthesis in Gram-positive bacteria.

The canonical pathway for heme synthesis uses protoporphyrinogen oxidase (PgoX) to convert coproporphyrinogen III (CPGIII) into protoporphyrinogen IX (PPGIX), but the divergent pathway characterized in Firmicutes and Actinobacteria does not have a protoporphyrin intermediate (8). These bacteria instead use coproporphyrinogen oxidase (CgoX) to convert CPGIII into coproporphyrin III (CPIII), which provides an alternate pathway and establishes CgoX as a potential antibacterial target that is unique to Gram-positive bacteria. CgoX and PgoX were both previously referred to as HemY, but were renamed due to the discovery of this divergence (8, 9).

Small molecule VU0038882 ('8882) was identified in a high throughput screen for activators of the S. aureus heme-sensing system (HssRS) two component system (10-12). Bacteria exposed to '8882 produce increased levels of heme leading to activation of HssRS (10-12). This increased heme production occurs through '8882-dependent activation of CgoX which provides increased amounts of CPIII and is specific to Gram-positive bacteria (10, 13).

In addition to producing its own heme, S. aureus expresses a set of proteins known as the iron-regulated surface determinant (Isd) system, that allow this organism to sequester iron from heme during iron starvation caused by the host during infection (14-17). The Isd proteins in S. aureus include the surface-exposed proteins IsdA and IsdB. Monoclonal antibodies (mAbs) to IsdA or IsdB have therapeutic efficacy in a septic murine model of S. aureus infection due to both heme-blocking and Fc-mediated effector functions (15, 18). These antigenic and biosynthetic differences between iron-starved Gram-positive bacteria and eukaryotes provide an opportunity to develop therapeutics targeting heme uptake and synthesis pathways in Gram-positive bacteria.

Photodynamic therapy (PDT) is a method that utilizes a photosensitive molecule (photosensitizer) activated by a specific wavelength of light to induce cell death through the production of reactive oxygen species (19). 5-Aminolevulinic acid (ALA) derivatives dominate the US Food and Drug Administration-approved photosensitizer market as a prodrug for the treatment of cancer (20). ALA is the first committed precursor in the heme biosynthesis pathway; therefore, heme biosynthesis increases when ALA is present in all species, including mammals. ALA leads to increased production of intermediates in the heme biosynthesis pathway, including photoactivators (19).

CPIII, a photoreactive tetrapyrrole intermediate of Gram-positive heme biosynthesis, can be targeted in antimicrobial photodynamic therapy (aPDT) with an excitation wavelength between 390 and 420 nm (21-23). However, the most effective photosensitizers for skin infections are likely those activated in the near infrared (NIR) parts of the spectrum due to high penetrance of near infrared light into the skin (24).

'8882-dependent activation of CgoX promotes CPIII accumulation in a similar manner to that of ALA. Furthermore, because this process is specific for Gram-positive bacteria, small-molecule activation of CgoX and subsequent treatment with PDT is a promising approach for skin and soft tissue skin infections, as it leaves host cells unaffected (25).

Disclosed herein is a method and system for inducing death or inhibiting growth or reproduction of a Gram-positive bacterial cell, which involves use of a combination of a CgoX activator, a photosensitizer conjugated to an Isd-targeting moiety, and multi-wavelength photodynamic therapy.

As used in the presently-disclosed subject matter, the CgoX activator promotes accumulation of CPIII in the target bacteria cell, and the CPIII serves as an intracellular photosensitizer having a first excitation wavelength.

The Iron-regulated surface-determinate (Isd) pathway that is present in a number of Gram-positive bacteria is harnessed in the presently-disclosed subject matter. Isd proteins are expressed on the surface of the bacterial cell. Thus, the photosensitizer conjugated to the Isd-targeting moiety is delivered to the bacterial cell, where it serves as an extracellular photosensitizer having a second excitation wavelength.

Multi-wavelength photodynamic therapy is employed in the presently-disclosed subject matter to deliver the first excitation wavelength and the second excitation wavelength. In this manner, photosensitizers are activated and impact the bacterial cell both intracellularly and extracellularly.

The excitation wavelength of the CPIII is in the range of about 390 nm to about 420 nm. However, the extracellular photosensitizer can be strategically selected with consideration to particular circumstances. For example, the extracellular photosensitizer can be selected to have an excitation wavelength that is beneficial in connection with the site of an infection by the bacterial cell. For a skin infection, for example, it could be beneficial to select a photosensitizer having an excitation wavelength in the red or near infrared parts of the spectrum, due to high penetrance of such wavelengths into the skin.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C. Photoactivator-conjugated (mAbs) kill iron-starved S. aureus strain Newman. Newman wild-type or mutant ΔisdAB Δspa bacterial burdens after treatment with antibody-dependent aPDT. Newman wild-type or mutant ΔisdAB Δspa, iron-starved using 1 mM dipyridyl, were exposed to anti-IsdA (FIG. 1A) and anti-isdB (FIG. 1B) monoclonal antibodies (Choby, et al, 2016)¹³ conjugated with the photosensitizer IR-700DX and tested for efficacy in the antimicrobial photodynamic therapy in vitro protocol. Newman wild-type or mutant ΔisdAB Δspa bacterial burdens after STAU-149 or STAU-281 treatment (FIG. 1C) were tested in combination for aPDT. Data presented as the averages of three independent experiments±SEM. Statistical significance was determined by one-way ANOVA with a Dunnet's test adjustment for multiple comparisons relative to “Vehicle” condition (FIGS. 1A and 1B), or Sidak's test adjustment for multiple comparisons relative to “Combo” condition (FIG. 1C) (****p≤0.0001, ***p≤0.001, **p≤0.01).

FIGS. 2A and 2B. VU0038882 analog activation of CgoX measured using phrt.xylE colorimetric assay. Activity of hrt promoter in response to VU0038882 and top-performing structural analogs, selected from high-throughput screen was quantified using Newman wild-type carrying the xylE reporter plasmid. Strains were grown in triplicate in the presence of vehicle, 10 μM (FIG. 2A) or 30 μM (FIG. 2B). XylE activity was quantified and normalized to protein concentration to determine specific activity. Analogs exhibiting toxicity to S. aureus are '9133 and '9952. Data presented are the average of eleven biological replicates±SD. Statistical significance was determined by one-way ANOVA with Dunnet's test adjustment for multiple comparisons (****p≤0.0001, ***p≤0.001, **p≤0.01, *p≤0.5, ns=not significant).

FIGS. 3A and 3B. The combination of small molecule and IR700DX-conjugated monoclonal antibody (mAb) treatments results in a greater reduction of CFUs/mL in vitro than either condition alone, independent of wild-type S. aureus strain tested. Enumeration of Newman wild-type or ΔisdAB Δspa exposed to anti-IsdA and anti-IsdB mAb mixture alone, VU0038882 alone, or the combination of the two conditions, with or without 50 J/cm² 395 nm and 63 J/cm² 690 nm light exposure (FIG. 3A). Enumeration of Newman wild-type, USA300 wild-type, U19 20, or U19 24 bacteria exposed to anti-IsdA and anti-IsdB mAb mixture alone, VU0038882 alone, or the combination of the two conditions, with or without 50 J/cm² 395 nm and 63 J/cm² 690 nm light exposure (FIG. 3B). Data presented as the averages of four independent experiments performed in duplicate±SEM. Statistical significance was determined by one-way ANOVA with Dunnet's test adjustment for multiple comparisons (****p≤0.0001, ***p≤0.001, **p≤0.01).

FIGS. 4A-4C. STAU-149 and STAU-281 bind to IsdA/B on the surface of iron-starved USA300. Super-resolution images depicting specificity of antibody binding to IsdA/B. USA300 wild-type or ΔisdAB Δspa were iron-starved prior to 30 min exposure to 149-IR700DX and 281-IR700DX or a vehicle control. Samples were stained with Hoechst and WGA and fixed for imaging via Nikon Structured Illumination Microscopy. Images shown in FIG. 4A represent 12 individual fields of view that were collected per sample. Total intensity of IR700DX staining normalized to Hoechst total intensity per image (FIG. 4B). Data presented are averages of 12 individual fields of view±SD. Statistical significance was determined using a two-way ANOVA with a Sidak's test adjustment for multiple comparisons (****p≤0.0001). Total intensity of IR700DX staining normalized to WGA total intensity per image (FIG. 4C). Data presented are averages of 12 individual fields of view±SD. Statistical significance was determined using a two-way ANOVA with a Sidak's test adjustment for multiple comparisons (****p≤0.0001).

FIGS. 5A and 5B. Photodynamic therapy using a combination of VU00038882 and photosensitizer conjugated (mAbs) results in a significant reduction in bacterial luminescence in vivo. FIG. 5A includes representative bioluminescence images obtained using the IVIS system prior to treatment or after the full two-day treatment regimen. In FIG. 5B, bacterial growth expressed as the percent difference of the integrated density of bioluminescent signal before or after the full treatment regimen. Data are presented as the mean of three (3) independent experiments±SEM. Statistical significance was determined using one-way ANOVA with Tukey's test adjustment for multiple comparisons (*p≤0.05).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods, compositions, kits, and systems for inhibiting growth of, treating an infection by, and/or treating a condition involving infection by a bacterial cell. The presently-disclosed subject matter provides effective alternatives to the use of traditional antibiotics, and has surprisingly-superior efficacy as compared to other known alternatives.

Some embodiments of the presently-disclosed subject matter include a method of treating an infection by a bacterial cell include contacting the bacterial cell with a coproporphyrinogen oxidase (CgoX) activator, thereby promoting accumulation of coproporphyrin III (CPIII) in the bacterial cell; contacting the bacterial cell with a targeting moiety conjugated to a photosensitizer, wherein the targeting moiety selectively binds iron-regulated surface determinant (Isd) proteins expressed on the surface of the bacterial cell; exposing the bacterial cell to light having a first excitation wavelength of CPIII; and exposing the bacterial cell to light having a second excitation wavelength of the photosensitizer that is conjugated to the antibody.

Some embodiments of the presently-disclosed subject matter include a method of inducing death or inhibiting growth or reproduction of a Gram-positive bacterial cell, which involves contacting the bacterial cell with a coproporphyrinogen oxidase (CgoX) activator, thereby promoting accumulation of coproporphyrin III (CPIII) in the bacterial cell; contacting the bacterial cell with a photosensitizer conjugated to an iron-regulated surface determinant (Isd) protein targeting moiety; exposing the bacterial cell to light having a first excitation wavelength of CPIII; and exposing the bacterial cell to light having a second excitation wavelength of the photosensitizer that is conjugated to the targeting moiety.

As noted herein, photodynamic therapy (PDT) is a tool that has shown some promise as an alternative to traditional antibiotics in the treatment of bacterial infection. A well-known example is the use of blue light and a photosensitizer to target bacteria that contributes to acne. However, known PDT methods have various shortcomings, including a lack of specificity for the target bacterial cell relative to surrounding host cells, and the relatively low penetration capacity of blue light is associated limitations in efficacy of treating infections occurring at locations beyond its penetration depth.

Due to the rise of antibiotic resistant bacteria, effective alternative treatments for infections are needed. The presently-disclosed subject matter has been developed to make use PDT, while overcoming shortcomings of known PDT methods, including, for example, specificity and efficacy issues.

The presently-disclosed subject matter is uniquely designed to specifically expose bacterial cells to intracellular and extracellular photosensitizers simultaneously, and also to expand penetration capacity of excitation wavelengths associated with the employed photosensitizers.

The presently-disclosed method includes contacting the bacterial cell with a coproporphyrinogen oxidase (CgoX) activator, thereby promoting accumulation of coproporphyrin III (CPIII). CPIII is a photoreactive heme intermediate (photosensitizer) having an excitation wavelength of about 390-420 nm. CgoX is specific to gram-positive bacteria. Thus, CgoX activation will target an infection-causing gram-positive bacterial cell, while leaving the host cells unaffected. In this manner, the presently-disclosed method specifically exposes bacterial cells to intracellular photosensitizers.

The presently-disclosed method also includes contacting the bacterial cell with a photosensitizer conjugated to a targeting moiety that selectively binds proteins that are expressed on the surface of the target bacterial cells. Iron-regulated surface determinant (Isd) proteins are expressed on the surface of gram-positive bacterial cells, such as Staphylococcus. In this manner, the presently-disclosed method specifically exposes bacterial cells to extracellular photosensitizers, while leaving the host cells unaffected.

Furthermore, the photosensitizer conjugated to the targeting moiety can be selected to have an excitation wavelength that has a desirable penetration capacity. Penetration depth of light into a material is a function of wavelength. In this regard, red light will generally have a greater penetration depth than blue light in a given material. In this manner, the presently-disclosed method provides a photosensitizer that can be selected to have an excitation wavelength that is greater than that of blue light, to enhance penetration capacity relative to blue light.

With the target bacterial cells simultaneously exposed to both intracellular and extracellular photosensitizers, the presently-disclosed method further involves exposing the bacterial cell to light having a first excitation wavelength directed to the intracellular photosensitizer, and a second excitation wavelength directed to the extracellular photosensitizer. In this manner, the presently-disclosed method provides a robust and targeted treatment for gram-positive bacterial infections, which is an effective alternative to traditional antibiotics and which achieves surprising and significantly improved results relative to known methods.

As noted, the presently-disclosed subject matter has particular utility in the context of targeting a gram-positive bacterial cell. In some embodiments of the method, the bacterial cell is selected from the group consisting of Staphylococcus, Streptococcus, Enterococcus, Bacillus, Clostridium, or Listeria. In some embodiments, the bacterial cell is Staphylococcus, for example, S. aureus.

As noted herein, the presently-disclosed subject matter also makes use of a CgoX activator, which promotes accumulation of CPIII in a target gram-positive bacteria cell. Examples of CgoX activators that can be used in connection with the presently-disclosed subject matter include the compounds identified in U.S. Pat. No. 9,867,879. In some embodiments, the CgoX activator can be a compound of the formula

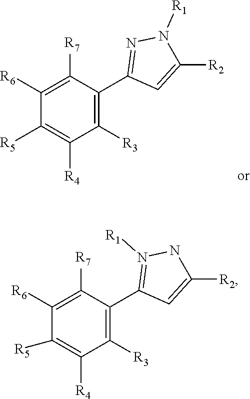

wherein, R₁ is H, alkyl, aryl, heteroaryl; R₂ is H, halogen, alkyl, aryl, heteroaryl; R₃ is H, hydroxyl, alkoxy, alkyl, aryl, heteroaryl, amino, amino sulfonyl, acetamide; R₄ is H, hydroxyl, alkoxy, alkyl, aryl, heteroaryl, amino, amino sulfonyl, acetamide; R₅ is H, hydroxyl, alkoxy, alkyl, aryl, heteroaryl, amino, amino sulfonyl, acetamide; R₆ is H, hydroxyl, alkoxy, alkyl, aryl, heteroaryl, amino, amino sulfonyl, acetamide; R₇ is H, hydroxyl, alkoxy, alkyl, aryl, heteroaryl, amino, amino sulfonyl, acetamide; and R₈ is —CR₃, O, S; wherein R₅ and R₆, R₇ and R₆, R₅ and R₄, R₄ and R₃ can cyclize forming a 3-10 member ring comprising C, O, S, and/or N optionally substituted with one or more R₃.

In some embodiments of the CgoX activator compound, R₁ is H, alkyl, aryl; R₂ is H, halogen, alkyl, aryl, heteroaryl; R₃ is H, alkyl, hydroxyl, alkoxy, amino, amino sulfonyl, acetamide, aryl, heteroaryl; R₄ is H, alkyl, hydroxyl, alkoxy, amino, amino sulfonyl, acetamide; R₅ is H, alkyl, hydroxyl, alkoxy, amino, amino sulfonyl, acetamide; and R₆ is H, alkyl, aryl.

In some embodiments of the CgoX activator compound, R₁ is H, CH₃, or

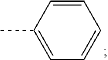

R₂ is H, CH₃, CH₂CH₃,

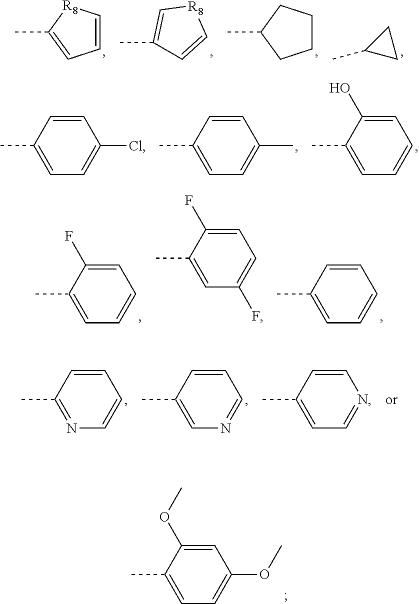

### R₃ is H, OH,

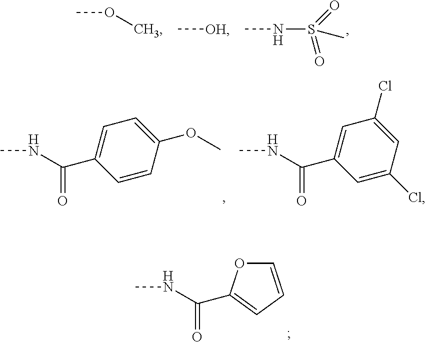

### R₄ is H, OH,

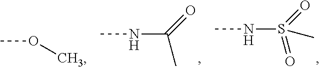

or R₄ and R₅, taken together with the carbon atoms to which they are bonded, can form an aromatic ring containing 6 carbon atoms; R₅ is H, OH,

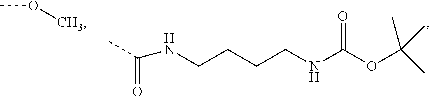

or R₄ and R₅, taken together with the carbon atoms to which they are bonded, can form an aromatic ring containing 6 carbon atoms, or R₅ and R₆, taken together with the carbon atoms to which they are bonded, can form an aromatic ring containing 6 carbon atoms; R₆ is H, or R₅ and R₆, taken together with the carbon atoms to which they are bonded, can form an aromatic ring containing 6 carbon atoms; R₇ is H or OH; and R₈ is O or S.

In some embodiments, the CgoX activator is selected from the compounds as set forth in Tables 2 and 3, hereinbelow.

As noted herein, the CgoX activator promotes accumulation of CPIII in the target bacterial cell. CPIII is photosensitizer having an excitation wavelength of about 390-420 nm. In this regard, in some embodiments, the presently-disclosed subject matter makes use of blue, violet, or ultraviolet A (UVA) light that includes a first excitation wavelength. In some embodiments, the presently-disclosed subject matter makes use of light including a first excitation wavelength is about 390 nm to about 420 nm.

The presently-disclosed subject matter also makes use of a photosensitizer conjugated to an iron-regulated surface determinant (Isd) protein targeting moiety. The Isd targeting moiety selectively binds Isd proteins expressed on the surface of a target bacterial cell.

In some embodiments, the targeting moiety selectively binds IsdA or IsdB. In some embodiments, the targeting moiety is an IsdA or an IsdB antibody. The antibody can be, for example, a monoclonal antibody. In some embodiments, the antibody is a human monoclonal antibody, such as, for example, an IsdA or an IsdB antibody as previously described (13), (15), (18), (50).

As described herein, the photosensitizer conjugated to the targeting moiety can facilitate therapeutic efficacy by being selected based on the penetration depth to reach a site of infection. In this regard, it can be desirable to select a photosensitizer having a second excitation wavelength that is greater than the first excitation wavelength associated with the CgoX activator. For example, in some embodiments, the photosensitizer is selected to have a second excitation wavelength is within red or near-infrared light.

In some embodiments, the photosensitizer is selected to have a second excitation wavelength of about 650 nm to about 1400 nm. In some embodiments, the photosensitizer is selected to have a second excitation wavelength of about 650 nm to about 800 nm. In some embodiments, the photosensitizer is selected to have a second excitation wavelength of about 680 nm to about 1400 nm. In some embodiments, the photosensitizer is selected to have a second excitation wavelength of about 700 nm to about 1200 nm. In some embodiments, the photosensitizer is selected to have a second excitation wavelength of about 690 nm.

It is contemplated that any known red or near infrared (NIR) dye could be selected for use as the photosensitizer of the presently-disclosed subject matter. Such useful dyes include, but are not limited to cyanines, indocyanine green (ICG), pyrrolopyrrole cyanines, phthalocyanines, porphyrins, squaraines, boron-dipyrromethanes, isobenzofuran-containing dyes, isothianaphthene-containing dyes, xanthenes, squaraines, polymethines, donor-acceptor-donor (D-A-D) dyes, zwitterionic dyes, and AIE luminogens (AIEgens). Examples of dyes that can be used in connection with the presently-disclosed subject matter include, but are not limited, to those described in, for example, Escobendo, et al. (2010) and Zhu, et al. (2019) (51), (52).

In some embodiments, the presently-disclosed subject matter is used for treating an infection in a host subject, such as an animal subject, for example, a human. The infection can be, for example, in skin or soft tissue of the animal.

In some embodiments, the CgoX activator and the Isd-targeting moiety-conjugated photosensitizer are separately provided for administration. In some embodiments, the CgoX activator and the Isd-targeting moiety-conjugated photosensitizer are each provided in a separate pharmaceutical composition, which further includes a pharmaceutically-acceptable carrier. In some embodiments, the CgoX activator and the Isd-targeting moiety-conjugated photosensitizer are provided together in a single composition. In some embodiments, the single composition is a pharmaceutical composition, which further includes a pharmaceutically-acceptable carrier. In some embodiments, the CgoX activator and the Isd-targeting moiety-conjugated photosensitizer are administered to the host topically. For example, administration can be provided topically to a location adjacent a skin or soft tissue infection.

The bacterial cell can be exposed to light having a first excitation wavelength and a second excitation wavelength. As will be recognized by the skilled artisan, an appropriate light source can be selected and/or adapted based on the first excitation wavelength and second excitation wavelength and the context in which the presently-disclosed subject matter is employed. For example, in the case of treating a skin or soft-tissue infection in a host animal, the bacterial cell can be exposed to the light by exposing the host to the light at a location adjacent the skin or soft-tissue infection. In some embodiments, the presently-disclosed subject matter is used to treat a condition associated with a bacterial infection, such as, for example, acne.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “ring” includes ring systems. For example, “ring” includes phenyl and napthyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹—(OA²)ₐ—OA³, where “a” is an integer of from 1 to 200 and A¹, A², and A³ are alkyl and/or cycloalkyl groups.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “acetamide” as used herein is represented by a formula A¹—NH—CO—A¹, or A¹—NH—CO—A¹—NH—CO—A¹, where A¹ is hydrogen or optionally substituted alkyl, cycloalkyl, alkenyl, cycloalkenyl, alkynyl, cycloalkynyl, aryl, or heteroaryl group as described herein.

The term “amino sulfonyl” as used herein is represented by a formula A¹—NH—SO₂—A¹, where A¹ is hydrogen or optionally substituted alkyl, cycloalkyl, alkenyl, cycloalkenyl, alkynyl, cycloalkynyl, aryl, or heteroaryl group as described herein.

As used herein, the terms “treatment” or “treating” relate to any treatment of a microbial infection including therapeutic, (i.e., post-infection), and prophylactic treatment, (i.e., pre-infection). As such, the terms treatment or treating include, but are not limited to: preventing a microbial infection or the development of a microbial infection; inhibiting the progression of a microbial infection; arresting or preventing the development of a microbial infection; reducing the severity of a microbial infection; ameliorating or relieving symptoms associated with a microbial infection; causing a regression of a microbial infection or an abscess or one or more of the symptoms associated with a microbial infection; and/or reducing the viability, infectivity and/or virulence of the bacteria. In some cases, in addition to making use of the methods described herein, it can be desirable to make use of traditional and other known treatment protocols. Although such traditional and other known treatment protocols will not be described in any detail herein, they will be known and understood by those skilled in the art, and it is contemplated that such traditional and other known treatment protocols can be used in combination with the methods and compositions described herein, if desired.

As used herein, the term “subject” refers to humans, and other animals. Thus, veterinary treatment is provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the treatment of livestock, including, but not limited to, domesticated swine, ruminants, ungulates, horses (including race horses), poultry, and the like.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “light therapy” and “photo therapy” are used interchangibly to refer to treatment that involves use of light. Various systems, devices, and techniques are known to those of ordinary skill in the art and appropriate selections will be apparent to those of ordinary skill in the art upon study of the present disclosure. Exemplary descriptions relevant to light therapy as contemplated in connection with the presently-disclosed subject matter include, but are not limited to: Fernadez, et al., (2010) “Two Coregulated Efflux Transporters Modulate Intracellular Heme and Protoporphyrin IX Availability in Streptococcus agalactiae,” PLOS Pathogens, 6 (4); Nakonieczna, et al., (2010) “Superoxide dismutase is upregulated in Staphylococcus aureus following protoporphyrin-mediated photodynamic inactivation and does not directly influence the response to photodynamic treatment,” BMC Microbiology, 10:323; Morimoto, et al, (2014), “Photodynamic Therapy Using Systemic Administration of 5-Aminolevulinic Acid and a 410-nm Wavelength Light-Emitting Diode for Methicillin-Resistant Staphylococcus aureus-Infected Ulcers in Mice,” PLOS ONE, 9 (8). Various photodynamic therapy (PDT) light sources can be used. Examples of PDT light sources are described in U.S. Pat. Nos. 8,175,687, 6,835,202, 6,645,230; and Patent Application Publication Nos. US2007/0233209 and WO2014/146029, each of which is incorporated herein by this reference. Exemplary light sources for PDT will also be known to those of ordinary skill in the art and include, for example the LEVULAN KARASTICK® (DUSA Pharmaceuticals, Inc.).

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Bacterial Strains, Growth Conditions, and Reagents

All bacterial strains used in this study are detailed in Table 1. Bacterial strains were grown in Difco tryptic soy broth (TSB; BD, Sparks, MD) at 37° C. with 180 rpm shaking. Metal starvation was achieved through growth in media containing 1 mM 2,2-dipyridyl. Strains containing phrt.XylE were grown in media containing 10 μg/mL chloramphenicol (11, 25). Lysis buffer was made by combining 100 mM potassium phosphate buffer with 10% by volume acetone and bringing to total volume with MilliQ water before adding 20 μg/mL lysostaphin. Assay buffer was potassium phosphate buffer made to a concentration of 100 mM and pH 8.0. VU0038882 and its analogs were generated by the combined efforts of Vanderbilt University Chemical Synthesis Core and Walter Reid Army Institute of Research.

TABLE 1
Strains

| | | | Reference(s) |
|---|---|---|---|
| Species | Genotype | Description | or source |
| S. aureus strain | WT | Wildtype laboratory stock | (49) |
| Newman | | | |
| S. aureus strain | phrt.XylE | XylE reporter strain | (11) |
| Newman | | | |
| S. aureus strain | ΔisdAB ΔspA | Triple mutant antibody control | (40) |
| Newman | | strain | |
| S. aureus strain | integrated | Clinically relevant strain with | (45) |
| USA300 NRS384 | luxBADCE | integrated luminescence cassette | |
| | reporter | for wound monitoring | |
| S. aureus strain | MLST: ST25 | Clinically isolated MSSA strain | This work |
| U19 20 - 2016 | | from VUMC patient SSTI | |
| S. aureus strain | MLST: ST72, | Clinically isolated MSSA strain | This work |
| U19 24 - 2016 | CC8 | from VUMC patient SSTI, | |
| | | necrotic tissue, and abscesses | |

### Example 2: Human Monoclonal Antibody (Mab) Conjugation to IR700DX Photosensitizer

Photosensitizer-antibody conjugates were synthesized using previously developed human mAbs that target the iron-regulated surface determinant (Isd) proteins of S. aureus (15, 18). A stock solution of the photosensitizer IR700DX-NHS ester (9 mM, LI-COR Biosciences) was prepared in DMSO. IR700DX-NHS ester was then mixed with antibodies at a molar ratio of 5:1, followed by addition of 0.1 mM Na₂HPO₄ (pH=8.4) to a total volume of 1 mL. After being stirred at room temperature for three hours, the mixture was purified using a Sephadex G-25 column (PD-10, GE Healthcare) and eluted with PBS (pH=7.4) in accordance with the manufacturer instructions.

The antibody concentration was determined using the Pierce™ BCA Protein Assay Kit (Thermo Scientific) and measuring the absorption at 562 nm, while the concentration of IR700DX was determined by measuring the absorption at 690 nm. These measurements allowed for the calculation of the average number of IR700DX molecules conjugated to each antibody. The purified photosensitizer-antibody conjugates were stored at −20° C. in 100 μL aliquots to reduce the number of freeze-thaw cycles to prevent decomposition. All antibodies in this study are conjugated with IR700DX.

### Example 3: Selection of Optimal Monoclonal Antibody Combination by Photodynamic Therapy

Iron-chelated bacterial cultures of the indicated strains were prepared. Antibodies of interest were added to 5 mL of iron-chelated TSB at a concentration of 2.5 ug/mL for subculture. These conditions were then separated into four-1 mL aliquots, which was comprised of replicates for both the experimental and control strains. The iron-depleted bacterial strains Newman wildtype and Newman ΔisdABΔspa (40) were then normalized to an OD₆₀₀ of 1.0 and back diluted 1:100 into each condition from the overnight cultures. These subcultures were then incubated at 37° C. and 180 rpm for 30 minutes, and the bacteria were pelleted via centrifugation for 5 minutes at 16,400 g and 4° C. The pellet was washed twice in 1 mL of cold PBS before being finally resuspended in 1 mL of cold PBS.

During the subculture incubation, a measurement was taken of the power of each light to calculate the time needed to achieve a light dose of 63 J/cm² for the 690 nm NIR light (M680L4; Thorlabs) and 50 J/cm² for the 395 nm blue-LED (M395L4; Thorlabs) mounted 5 cm above the measurement probe. This measurement was taken using a photodiode and power meter (919P-003-10, 843-R; Newport) with a cutout 3×3 portion of a black 384-well microtiter plate positioned centrally over the measurement probe. The outer wells were covered with aluminum foil to ensure that the measurement taken by the probe was strictly limited to the light that entered the 0.1225 cm² area of the well. This approach allowed for the irradiance of the light to be calculated according to (readout (mW)/0.1225 cm²). This value was then divided into the total light dose for each light and divided by 60 to determine the light dose in minutes. The outputs of the 690 nm and 395 nm LEDs were spatially combined using a long-pass dichroic filter (FF470-Dio; Semrock Inc.) for all experimental conditions where light was present.

The spatial intensity profile of the light output is approximately Gaussian in shape in both directions, so 25 μL of each sample was aliquoted into a 3×3 square on a black 384-well microtiter plate with the center well remaining empty, since it would receive a stronger dose than the surrounding wells. The samples were organized such that the conditions alternated wild-type and knockout in a clockwise fashion, beginning with the lower left corner well. In addition, the center well was centered under the light to equate the radial diminishment of light dose amongst the samples.

### Example 4: Screening of VU0038882 Analogs

To maximize the production of coproporphyrin III and thereby increase blue-light mediated killing of S. aureus, synthesized analogs of VU0038882 were tested. To determine the relative efficacy of each analog, a Newman strain of S. aureus, transformed with the phrt.xylE (11) reporter vector was used. This reporter allows for colorimetric assessment of intracellular heme production. To perform the assay, each individual analog was added to 3 mL of TSB+10 μg/mL chloramphenicol to reach a concentration of 30 μM, and then 700 μL of this newly made 30 μM solution was added to 1.3 mL of remaining TSB+10 μg/mL chloramphenicol media to create the 10 μM concentration. Either 10 μM or 30 μM concentrations of compounds in medium were then aliquoted into three 0.5 mL replicate tubes. Three separate 16- to 18-hour overnight subcultures from isolated colonies were back-diluted 1:100 into the tubes creating three biological replicates and were subcultured for 6 hours at 37° C. and 180 rpm. After incubation, the cells were pelleted at 16,400 g for 5 minutes and the supernatant aspirated before washing with 0.5 mL of 20 mM potassium phosphate wash buffer. After pelleting the cells again at 16,400 g for 5 minutes the wash buffer was aspirated, and the pellets were stored at −80° C. until further use.

When the cells were ready to be lysed and data collected, samples were removed from the −80° C. freezer, allowed to thaw, and resuspended in 150 μL of lysis buffer (100 mM potassium phosphate pH=8.0, 10% v/v acetone, 20 μg/mL lysostaphin) before incubation in a 37° C. bead bath for 20 minutes and then cooled for 5 minutes on ice. The samples were then centrifuged at 16,400 g and 4° C. for 30 minutes to separate the pellet from the lysate, which was then placed into a 96-well microtiter plate and kept on ice. The lysate from each sample was pipetted into 20 μL aliquots on a round-bottom or flat-bottom 96-well plate on ice. The round bottom plate was labeled for bicinchoninic acid assay (BCA) protein content analysis, and 10 μL of BCA standards was added to extra wells on the plate. Protein quantification was performed using a Pierce BCA Protein Assay Kit as per the manufacturer's instructions. While the BCA plate was in the incubator, the flat-bottom plate was prepared by thawing 1 M catechol on ice and making 100 mM potassium phosphate (pH=8.0) assay buffer. After preparing a kinetic read at 375 nm for 10 minutes with a minimal interval between reads, the catechol was diluted 1:1,000 into the assay buffer and quickly dispensed into a sterile basin, then 200 μL of the solution was pipetted into each well using a multichannel pipette. All data were exported into Excel software (Microsoft) and analyzed for specific activity of each compound and activity relative to VU0038882 controls.

After 219 analogs had been screened, the compounds with the best percent VU0038882 activity were tested again following the same protocol, but with twelve replicates per concentration with data expressed as averages ±the standard deviation (SD). Additionally, the top-performing compounds were tested using the XylE protocol with a concentration gradient to determine their half maximal effective concentration (EC50) values.

TABLE 2
Top performing VU0038882 analog EC₅₀ values.

| Compound | Structure | logEC50 | EC50 (μM) |
|---|---|---|---|
| ′9952 | 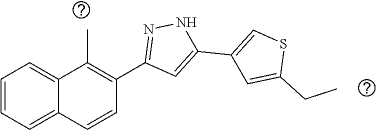 [SMILES: CCc1cc(-c2cc(-c3ccc4ccccc4c3C)n[nH]2)cs1] | −5.182 ± 0.065 | 6.58 |
| ′9133 | 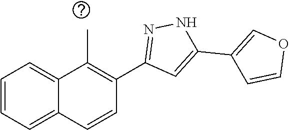 [SMILES: Cc1c(-c2cc(-c3ccoc3)[nH]n2)ccc2ccccc12] | −5.088 ± 0.023 | 8.17 |
| ′6700 | 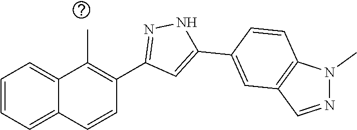 [SMILES: Cc1c(-c2cc(-c3ccc4c(cnn4C)c3)[nH]n2)ccc2ccccc12] | −5.087 ± 0.063 | 8.18 |
| ′8882 | 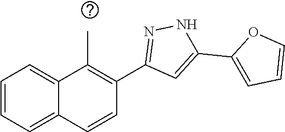 [SMILES: Cc1c(-c2cc(-c3ccco3)[nH]n2)ccc2ccccc12] | −4.978 ± 0.047 | 10.5 |
| ′9625 | 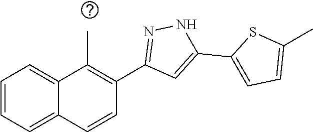 [SMILES: Cc1ccc(-c2cc(-c3ccc4ccccc4c3C)n[nH]2)s1] | −4.882 ± 0.055 | 13.1 |
| ′9626 | 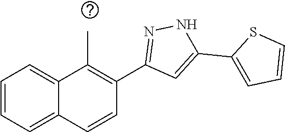 [SMILES: Cc1c(-c2cc(-c3cccs3)[nH]n2)ccc2ccccc12] | −4.701 ± 0.023 | 19.9 |
| ′4083 | 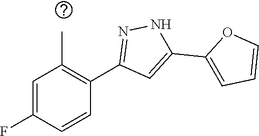 [SMILES: Cc1cc(F)ccc1-c1cc(-c2ccco2)[nH]n1] | −4.68 ±0.038 | 20.9 |
| ′9132 | 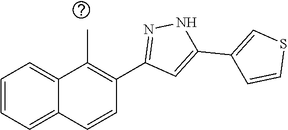 [SMILES: Cc1c(-c2cc(-c3ccsc3)[nH]n2)ccc2ccccc12] | −4.582 ± 0.075 | 26.2 |
| ′2069 | 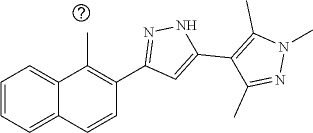 [SMILES: Cc1nn(C)c(C)c1-c1cc(-c2ccc3ccccc3c2C)n[nH]1] | −4.545 ± 0.098 | 28.5 |

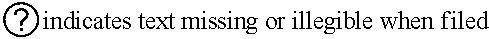

After assembling the list of optimal compounds from the XylE screen, each compound was tested for efficacy in the photodynamic killing described previously. As shown by the EC50 values of each of the compounds, the optimal concentration was near to 30 μM (Table 2), so that was the tested condition in light killing. To ensure the compounds' efficacy in the full therapeutic regimen and reduce potential unknown variables, each of the compounds was tested with 395 nm or 690 nm wavelengths and with Newman WT or Newman ΔisdAB Δspa.

### Example 5: In Vitro Toxicity Assessment in HepG2 Cells

Cytotoxicity was measured using a colorimetric assay with 3-(4,5-dimethylthiazol-2-yl)-2,5-diphenyltetrazolium bromide (Cell Proliferation Kit I, MTT; Roche)(41). Human hepatocarcinoma cells (HepG2) were cultured in complete Minimal Essential Medium (MEM from Gibco-Invitrogen, No.11090-099) prepared by supplementing MEM with 0.19% sodium bicarbonate (Gibco-BRL No. 25080-094), 10% heat inactivated fetal bovine serum (FBS; Gibco-Invitrogen No. 16000-036), 2 mM L-glutamine (Gibco-Invitrogen No. 25030-081), 0.1 mM MEM nonessential amino acids (Gibco-Invitrogen No. 11140-050), 0.009 mg/mL insulin (Sigma No. I1882), 1.76 mg/mL bovine serum albumin (Sigma No. A1470). Cell viability was assessed using the trypan blue exclusion method(42).

Using 96 well plates, HepG2 cells (2.5×10⁴) in 170 μL of culture medium were added to each well and incubated at 37° C. overnight in a humidified 5% CO₂ atmosphere. Drug plates were prepared with a Biomek 4000 automated laboratory station. Positive control was 4-azaindole[2,1-b]quinazoline-6,12-dione (43), and negative control was 0.2% DMSO. Test compounds or the positive control were suspended in DMSO and dispensed into 96 well plates at 1.6-fold serial dilutions for a final concentration range of 10 μg/mL to 0.15 μg/mL. Aliquots (30 μL) of each dilution and the negative control were then added to the 170 μL volume of cells and medium per well. Mixtures of sample and cells were incubated at 37° C. for 48 hours. After incubation, 30 μL of a solution of MTT (1.5 mg/mL) dissolved in complete MEM medium was added to each well, and plates were subsequently incubated in the dark for 1 h at room temperature. At this time, the medium and drugs in each well were removed, and plates were then left to dry in a laminar flow hood for 15 minutes. Formazan dye crystals were dissolved upon addition of acidified isopropyl alcohol (60 μL). Plates were placed on a 3-D rotator for 15-30 min before absorbance was measured at 590 nm using a Perkin Elmer EnSight plate reader. Half-maximal inhibitory concentration (IC₅₀) values were determined using Prism software (GraphPad) using the nonlinear regression equation (sigmoidal dose-response, variable slope). IC₅₀ values were reported in units of ng/mL. All samples were run in duplicate.

### Example 6: In Vitro Full Photodynamic Therapeutic Regimen

After determining the optimal mAb combination via the photodynamic killing assay and the most effective compounds via the XylE specific activity assay, the full therapeutic regimen was tested in vitro to ensure that the combination of antibodies and compounds was significantly more effective than either alone. Both VU0038882 ('8882) and VU0829626-1 ('9626) were tested with the antibody combination of STAU-149+STAU-281.

### Example 7: In Vitro Structured Illumination Microscopy

USA300 wild-type and Newman ΔisdABΔspa bacterial strains were grown overnight under iron-restricted conditions as previously described. Cultures were normalized to an OD600 ₙₘ of 0.5 and then exposed to vehicle or 125 μg/mL STAU-149 and STAU-281 for 30 minutes at 37° C. Cells were then collected and washed prior to staining with Hoechst 33342 (Invitrogen) and wheat germ agglutinin Alexa Fluor 488 (WGA; Thermo Fisher Scientific) for 5 minutes prior to fixation. Cells were fixed in 4% paraformaldehyde at room temperature for 30 minutes before being diluted and dried overnight on coverslips which were affixed to slides using Prolong Gold (Invitrogen). Slides were viewed using the 3D-SIM mode on a Nikon N-SIM structured illumination platform and images collected via an Andor DU-897 EMCCD camera. All samples were imaged 12 times with at least 100 bacterial cells per image. Images were analyzed using the Nikon NIS-Elements AR software. Imaging experiments and data analysis were performed in part through the use of the Vanderbilt Cell Imaging Shared Resource.

### Example 8: In Vivo Murine Skin Infection Model

All mice were maintained in compliance with Vanderbilt's Institutional Animal Care and Use Committee regulations (protocol M1800026). To create a superficial skin wound a similar model of infection was used, as previously described (44), with modifications to relocate the area of infection to the inner epithelial layer of each ear. Eight- to 10-week-old female BALB/cJ mice were used for the S. aureus infections. Twenty-four hours prior to infection the mice were injected intraperitoneally with 20 μg/mL of both STAU-149 (anti-IsdA) and STAU-281 (anti-IsdB) in PBS. Overnight cultures of S. aureus USA300 with a chromosomally-integrated luxBADCE cassette (45) were subcultured 1:50 into TSB containing 1 mM 2,2-dipyridyl. Cultures were grown for 3.5 h to reach exponential phase of growth. Cell pellets were washed twice with ice-cold PBS with the original culture volume, centrifuged, and resuspended in 250 μL of ice-cold PBS. Approximately 5×10⁷ log-phase CFUs were inoculated in 5 μL of PBS directly onto the surface of the wound present on each ear, and each mouse was again injected intraperitoneally with antibodies. Mice were placed into new sterile cages in groups of three in accordance with their experimental condition. Four hours post-infection, the mice were visualized using the IVIS spectrum (Perkin Elmer) in vivo bioluminescence imaging system, with data being collected with a 5-minute exposure time. Each mouse was then transferred to a nose cone under continuous isoflurane anesthesia and, 30 minutes prior to light exposure, the ears were exposed to 10 ∥L of the 450 ∥M '8882 and 20 μg/mL mAb cocktail in PBS containing 10% DMSO. Control mice were left untreated. The right ventrodorsal ear of each mouse was exposed to the 395-nm LED for 20.6-23.1 minutes delivering a total energy dose of 50 J/cm². Simultaneously, the same ear was exposed to the 690-nm LED for 57.3-60.5 minutes delivering a total energy dose of 63 J/cm².

The left ventrodorsal ear of each mouse was not exposed to light during treatment. Each mouse was transferred back to a cage to recover overnight. Twenty-four hours post-infection, the groups of mice were imaged again using the IVIS system. Luminescence was quantified for individual ears using ImageJ (FIJI) (46-48). After acquiring images, the mice were treated with 10 μL STAU-149+STAU-281 topically as previously described at a concentration of 20 μg/mL and exposed to 690 nm light alone for 56.9-60.0 minutes to a total energy dose of 63 J/cm². Post-treatment images were again collected at 5-minute exposure times using the IVIS system and analyzed using ImageJ (FIJI). After collecting the final set of images, the mice were sacrificed.

### Example 9: General Chemistry

All chemical reagents and reaction solvents were purchased from commercial suppliers and used as received. All microwave-assisted reactions were performed using a Biotage Initiator microwave reactor. Hydrogenation reactions are performed using an atmospheric balloon or using a Parr hydrogenation shaker apparatus where stated. Analytical thin-layer chromatography (TLC) was performed on Kieselgel 60 F254 glass plates precoated with a 0.25 mm thick silica gel. TLC plates were visualized with UV light and iodine. Normal phase flash silica gel-based column chromatography is performed using ready-to-connect cartridges from ISCO using a Teledyne ISCO Combiflash Rf+system.

All compounds were obtained at 95% purity or higher, unless otherwise noted, as measured by analytical reversed-phase HPLC performed on a Shimadzu Prominence LC-20AD series system with UV detection at 254 and 312 nm using the following method: Waters X-Select® CSHTM C18, 130 ∈, 5 μm LC column (4.6×100 mm) with a 15 min gradient of 5-95% CH3CN in H2O and 0.1% TFA.

For LCMS characterization of the compounds in the present invention, they were performed on a Waters Acquity UPLC H-Class System and one of the following methods were used: Method A: A Waters Acquity 130 ∈, 1.7 μm, UPLC BEH C18 column (2.1×50 mm) was used with a 4 min gradient of 5-95% CH3CN in H2O and 0.1% TFA. Method B: A Waters Acquity 130 ∈, 1.7 μm, UPLC BEH C18 column (2.1×50 mm) was used with an 8 min gradient of 5-95% MeCN in H2O and 0.1% TFA.

Proton nuclear magnetic resonance (1H NMR) spectra were recorded at either 400 or 600 MHz on a Bruker spectrometer, as stated. For 1H NMR spectra, chemical shifts are reported in parts per million (ppm) relative to residual nondeuterated solvent signals. Coupling constants are reported in hertz (Hz). The following abbreviations (or a combination thereof) are used to describe splitting patterns: s, singlet; d, doublet; t, triplet; q, quartet; pent, pentet; hept, heptet m, multiplet; br, broad.

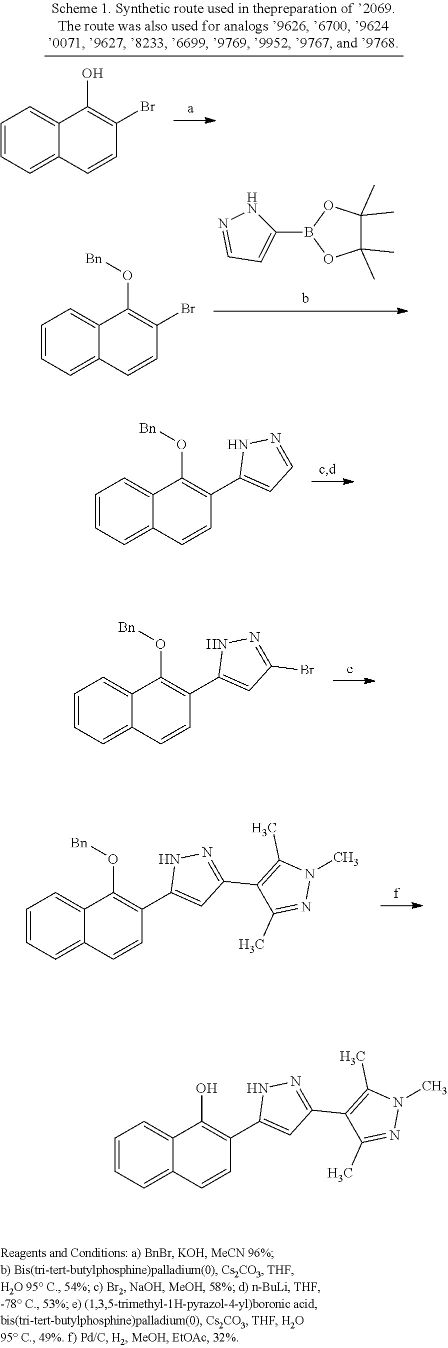

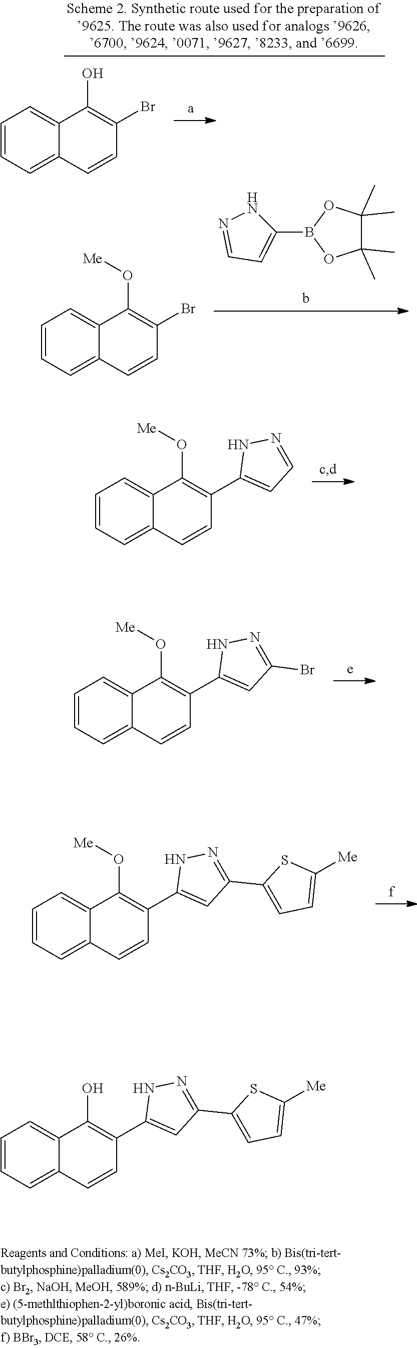

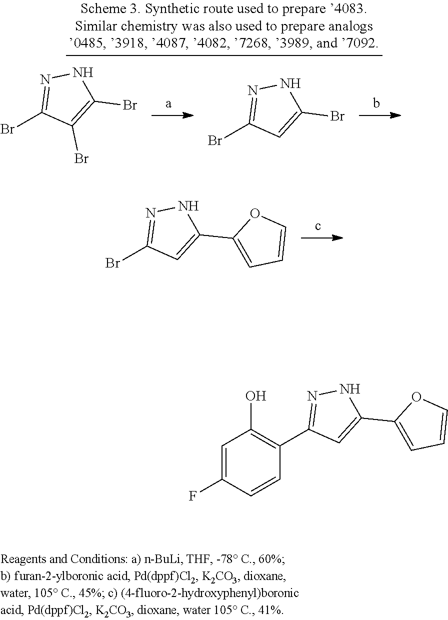 [SMILES: Brc1n[nH]c(Br)c1Br ; Brc1cc(Br)[nH]n1 ; Brc1cc(-c2ccco2)[nH]n1 ; Oc1cc(F)ccc1-c1cc(-c2ccco2)[nH]n1]

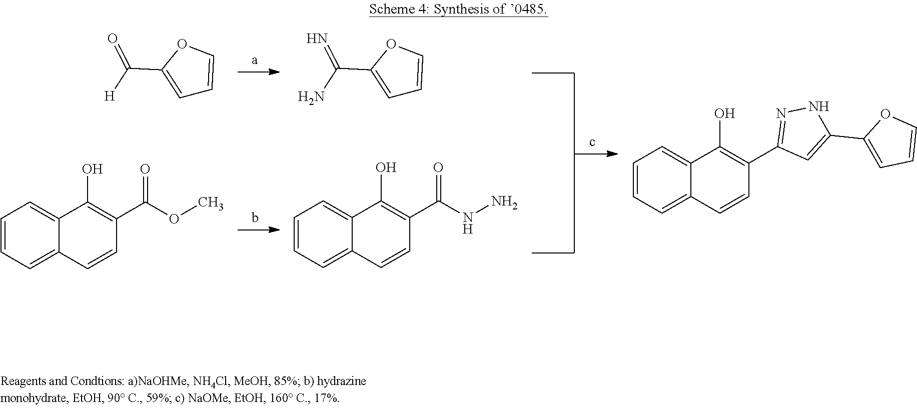 [SMILES: O=Cc1ccco1 ; N=C(N)c1ccco1 ; COC(=O)c1ccc2ccccc2c1O ; NNC(=O)c1ccc2ccccc2c1O ; Oc1c(-c2cc(-c3ccco3)[nH]n2)ccc2ccccc12]

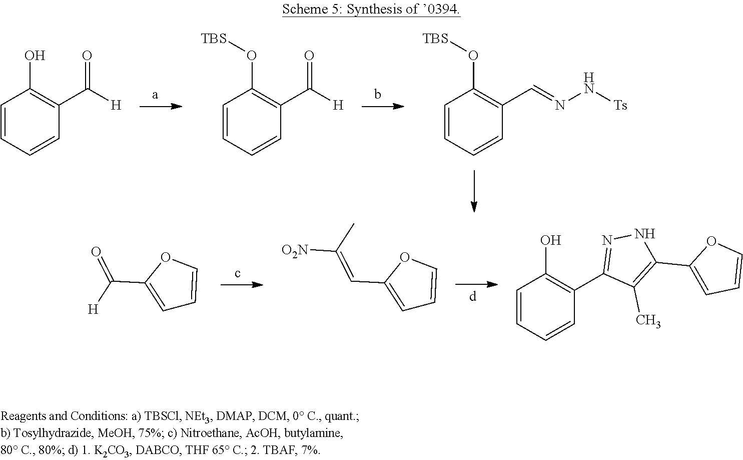

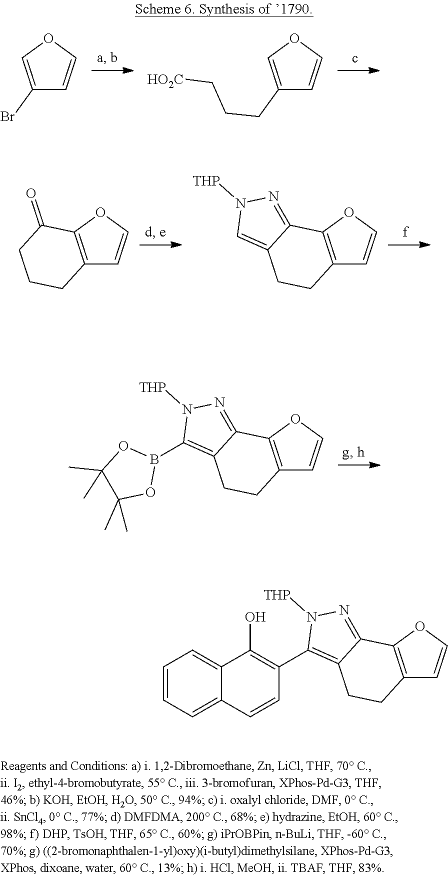

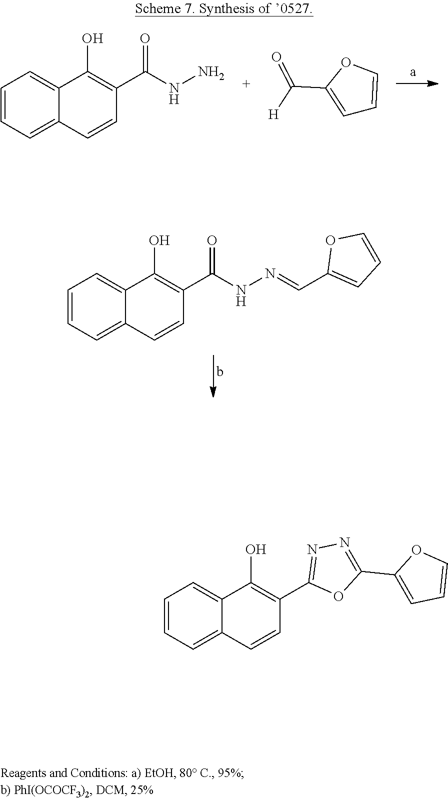 [SMILES: NNC(=O)c1ccc2ccccc2c1O ; O=Cc1ccco1 ; O=C(N/N=C/c1ccco1)c1ccc2ccccc2c1O ; Oc1c(-c2nnc(-c3ccco3)o2)ccc2ccccc12]

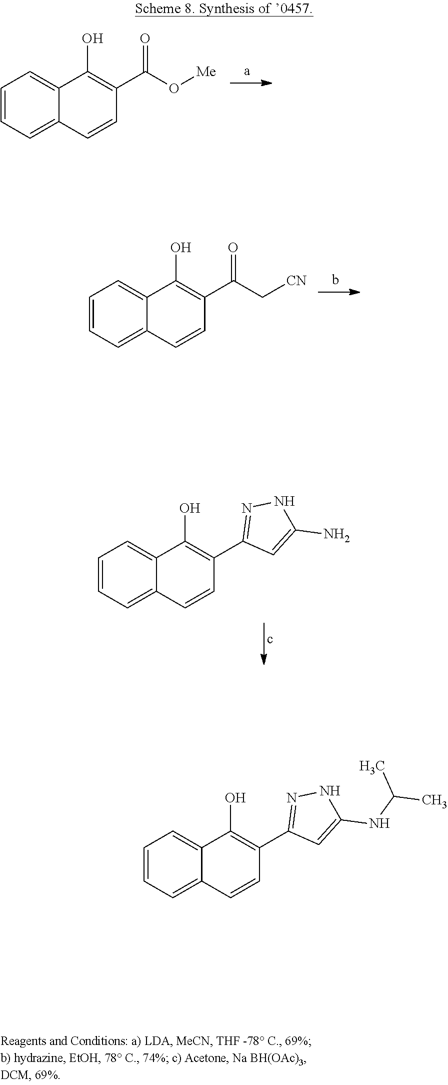 [SMILES: COC(=O)c1ccc2ccccc2c1O ; N#CCC(=O)c1ccc2ccccc2c1O ; Nc1cc(-c2ccc3ccccc3c2O)n[nH]1 ; CC(C)Nc1cc(-c2ccc3ccccc3c2O)n[nH]1]

### Example 10: Procedural Details of Synthesis of '8882: 2-(5-(Furan-2-yl)-1H-pyrazol-3-yl)naphthalen-1-ol

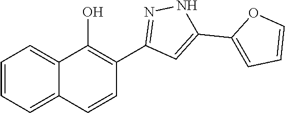 [SMILES: Oc1c(-c2cc(-c3ccco3)[nH]n2)ccc2ccccc12]

The synthesis of this molecule has been previously reported (10).

### Example 11: Procedural Details of Synthesis of '2069: 2-(1′,3′,5′-trimethyl-1′H,2H-[3,4′-bipyrazol]-5-yl)naphthalen-1-ol

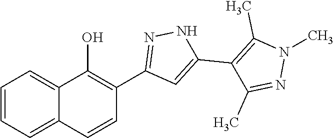 [SMILES: Cc1nn(C)c(C)c1-c1cc(-c2ccc3ccccc3c2O)n[nH]1]

1-(Benzyloxy)-2-bromonaphthalene. To a solution containing 2-bromonaphthalen-1-ol (2.0 g, 8.9 mmol) and 30 mL of MeCN was added KOH (1.0 g, 17.93 mmol). The reaction vessel was purged with Ar and benzyl bromide (4.26 mL, 35.86 mmol) was added over 10 minutes. The reaction mixture was allowed to stir at room temperature for 16 h, diluted with H₂O (30 mL), and extracted with EtOAc (3×, 30 mL). The combined organic layers were washed with brine, dried over MgSO₄, and concentrated under reduced pressure. The crude product was purified by flash chromatography using EtOAc/Hexane (0-40%) as eluents to afford 1-(benzyloxy)-2-bromonaphthalene (2.71 g, 96%). ¹H NMR (400 MHz, CDCl₃) δ (ppm) 8.22-8.8.18 (m, 1H), 7.90.-7.7.86 (m, 1H) 7.72-7.68 (m, 2H), 7.67 (s, 1H), 7.60-7.55 (m, 3H), 7.54-7.50 (m, 2H), 7.48-7.43 (m, 1H), 5.20 (s, 2H); LCMS (ESI) R_{T}=1.399 min, m/z=313.2 [M+H]⁺.

3-(1-(Benzyloxy)naphthalen-2-yl)-1H-pyrazole. To a solution containing 1-(benzyloxy)-2-bromonaphthalene (2.7 g, 8.6 mmol) and THF (30 mL) was added bis(tri-tert-butylphosphine)-palladium(0) (70 mg, 0.13 mmol) and 5-(4,4,5,5-tetramethyl-1,3,2-dioxaborolan-2-yl)-1H-pyrazole (1.8 g, 9.4 mmol). The reaction mixture was degassed for 30 minutes and a solution of Cs₂CO₃ (aq) (9.0 mL, 9 mmol) was added. The reaction mixture was heated at 95° C. for 16 h, allowed to cool to rt, filtered over a celite plug, and concentrated under reduced pressure. The crude product was purified by flash chromatography using EtOAc/Hexane (0-40%) as eluents to afford 3-(1-(benzyloxy)naphthalen-2-yl)-1H-pyrazole (1.4 g, 54%). ¹H NMR (400 MHz, CDCl₃) δ (ppm) 8.18-8.14 (m, 1H), 7.89-7.85 (m, 1H), 7.68-7.64 (m, 3H), 7.58-7.52 (m, 3H), 7.51-7.41 (m, 4H), 5.17 (s, 2H); LCMS (ESI) R_{T}=1.221 min, m/z=301.2 [M+H]⁺.

3-(1-(Benzyloxy)naphthalen-2-yl)-4,5-dibromo-1H-pyrazole. To a solution containing (1-(benzyloxy)naphthalen-2-yl)-1H-pyrazole (1.4 g, 4.6 mmol) and MeOH (20 mL) was added NaOH (1 M, aq) (14 mL, 14 mmol) and Br₂ (1.5 mL, 28 mmol). The reaction mixture was allowed to stir at rt for 16 h and concentrated under reduced pressure. The crude product was purified by flash chromatography using EtOAc/Hexane (0-70%) as eluents to afford 3-(1-(benzyloxy)naphthalen-2-yl)-4,5-dibromo-1H-pyrazole (1.2 g, 58%). ¹H NMR (400 MHz, CDCl₃) δ (ppm) 8.31-8.23 (m, 2H), 7.95-7.91 (m, 2H), 7.78 (d, J=8.7 Hz, 1H), 7.64-7.61 (m, 2H), 7.37-7.34 (m, 3H), 7.27-7.23 (m, 2H), 4.89 (s, 2H). LCMS (ESI) R_{T}=1.415 min, 537.1 m/z=[M+H+HBr]⁺.

3-(1-(Benzyloxy)naphthalen-2-yl)-5-bromo-1H-pyrazole. A solution containing 3-(1-(benzyloxy)naphthalen-2-yl)-4,5-dibromo-1H-pyrazole (5.1 g, 9.53 mmol) and THF (200 mL) was cooled to −78° C. n-BuLi (24 mL, 38.1 mmol) was added dropwise over 2.5 h, maintaining temperature at −78° C., and the reaction mixture was allowed to stir at this temperature for a further 1.5 h. MeOH (30 mL) was added slowly and the mixture was allowed to warm to rt over the next 16 h. The crude reaction was concentrated under reduced pressure and purified by flash chromatography using EtOAc/Hexane (0-40%) as eluents to afford 3-(1-(benzyloxy)naphthalen-2-yl)-5-bromo-1H-pyrazole (1.9 g, 53%). ¹H NMR (400 MHz, CDCl₃) δ (ppm) 8.26 (d, J=7.9 Hz, 1H), 7.91 (d, J=7.0 Hz, 1H), 7.75 (d, J=8.5 Hz, 1H), 7.64-7.57 (m, 3H), 7.52-7.43 (m, 5H), 6.70 (s, 1H), 5.00 (s, 2H). LCMS (ESI) R_{T}=1.219 min, m/z=379.2 [M+H]⁺.

Regioisomer 3-(1-(benzyloxy)naphthalen-2-yl)-4-bromo-1H-pyrazole was isolated as a minor product (0.49 g, 14%): ¹H NMR (400 MHz, CDCl₃) δ (ppm) 8.19-7.15 (m, 1H), 7.87-7.80 (m, 2H), 7.66 (d, J=8.4 Hz, 1H), 7.58 (s, 1H) 7.51-7.47 (m, 2H), 7.30-7.24 (m, 3H), 7.21-7.18 (m, 2H), 4.71 (s, 2H). LCMS (ESI) R_{T}=1.167 min, m/z=379.2 [M+H]⁺.

5-(1-(Benzyloxy)naphthalen-2-yl)-1′,3′,5′-trimethyl-1′H,2H-3,4′-bipyrazole. To a solution containing 3-(1-(benzyloxy)naphthalen-2-yl)-5-bromo-1H-pyrazole (75 mg, 0.20 mmol) and THF (5.0 mL) was added (1,3,5-trimethyl-1H-pyrazol-4-yl)boronic acid (62.0 mg, 0.30 mmol) and bis(tri-tert-butylphosphine)-palladium(0), (10.1 mg, 0.020 mmol). The reaction mixture was degassed for 15 min, and Cs₂CO₃ (aq, 1.0 M) (0.4 mL) was added. The reaction mixture was heated at 95° C. for 16 h, then cooled to rt. and absorbed on celite. The crude product was purified by flash chromatography using EtOAc/Hexane as eluents (0-90%) to afford 5-(1-(benzyloxy)naphthalen-2-yl)-1′,3′,5′-trimethyl-1′H,2H-3,4′-bipyrazole (40.4 mg, 49%). LCMS (ESI): R_{T}=1.111 min, m/z=409.4 [M+H]⁺.

2-(1′,3′,5′-Trimethyl-1′H,2H-[3,4′-bipyrazol]-5-yl)naphthalen-1-ol. A mixture containing 5-(1-(benzyloxy)naphthalen-2-yl)-1′,3′,5′-trimethyl-1′H,2H-3,4′-bipyrazole (40 mg, 0.097 mmol), 6 mL of a 1:1 mixture of MeOH:EtOAc (6 mL), and Pd/C (10%, 10 mg) was allowed to stir 16 h under an atmosphere of H₂. The mixture was filtered over a celite plug and the plug was further washed with MeOH. The crude product was purified by HPLC using 1% TFA (Aq)/MeCN as eluents to afford 2-(1′,3′,5′-trimethyl-1′H,2H-[3,4′-bipyrazol]-5-yl)naphthalen-1-ol '2069 (9.9 mg, 32%). ¹H NMR (400 MHz, CDCl₃) δ (ppm) 8.46-8.43 (m, 1H), 7.82-7.79 (m, 1H) 7.71 (d, J=8.6 Hz, 2H), 7.54-7.50 (m, 2H), 7.43 (d, J=8.5 Hz, 1H), 6.69 (s, 1H), 3.85 (s, 3H), 2.40 (s, 3H), 2.38 (s, 3H); LCMS (ESI) R_{T}=1.070 min, m/z=319.3 [M+H]⁺.

### Example 12: Procedural Details of Synthesis of '9133: 2-(5-(Furan-3-yl)-1H-pyrazol-3-yl)naphthalen-1-ol

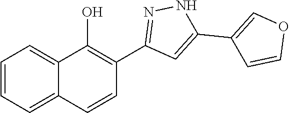 [SMILES: Oc1c(-c2cc(-c3ccoc3)[nH]n2)ccc2ccccc12]

3-(1-(Benzyloxy)naphthalen-2-yl)-5-(furan-3-yl)-1H-pyrazole. To a solution containing 3-(1-(Benzyloxy)naphthalen-2-yl)-5-bromo-1H-pyrazole (75 mg, 0.20 mmol) and THF (5.0 mL) was added furan-3-ylboronic acid (28.7 mg, 0.30 mmol), followed by bis(tri-tert-butylphosphine)-palladium(0) (10.1 mg, 0.020 mmol). This solution was degassed for 15 min and Cs₂CO₃ (aq, 1.0 M) (0.4 mL) was added. The reaction mixture was heated at 95° C. for 16 h, then cooled to rt and absorbed on celite. The crude product was purified by flash chromatography using EtOAc/Hexane as eluent (0-90%) to afford 3-(1-(benzyloxy)naphthalen-2-yl)-5-(furan-3-yl)-1H-pyrazole (88.3 mg, 91%). LCMS (ESI): R_{T}=1.235 min, m/z=367.3 [M+H]⁺.

2-(5-(Furan-3-yl)-1H-pyrazol-3-yl)naphthalen-1-ol. To a solution containing 3-(1-(benzyloxy)naphthalen-2-yl)-5-(furan-3-yl)-1H-pyrazole (75 mg, 0.20 mmol) and DCM (8 mL) at −78 ° C. was added a solution of BBr₃ (1.0 M in DCM) (0.4 mL, 0.4 mmol). The reaction mixture was allowed to stir at −78° C. for 2 h. At this time, MeOH (5.0 mL) was added and the mixture was allowed to slowly warm to rt and concentrated under reduced pressure. The crude product was purified by HPLC using 1% TFA (Aq)/MeCN as eluents to afford 2-(5-(furan-3-yl)-1H-pyrazol-3-yl)naphthalen-1-ol '9133 (13.1 mg, 23%). ¹H NMR (400 MHz, CDCl₃) δ (ppm) 8.49-8.45 (m, 1H), 7.89 (s, 1H), 7.83-7.79 (m, 1H), 7.69 (d, J=8.5 Hz, 1H), 7.59 (s, 1H), 7.55-7.50 (m, 2H), 7.44 (d, J=8.7 Hz, 1H), 7.39 (s, 1H), 6.86 (s, 1H), 6.74 (bs, 1H). LCMS (ESI) R_{T}=1.197 min, m/z=277.2 [M+H]⁺.

### Example 13: Procedural Details of Synthesis of '9132: 2-(5-(thiophen-3-yl)-1H-pyrazol-3-yl)naphthalen-1-ol

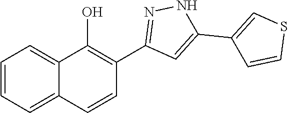 [SMILES: Oc1c(-c2cc(-c3ccsc3)[nH]n2)ccc2ccccc12]

3-(1-(Benzyloxy)naphthalen-2-yl)-5-(thiophen-3-yl)-1H-pyrazole: Using a procedure similar to that used in the preparation of 3-(1-(benzyloxy)naphthalen-2-yl)-5-(furan-3-yl)-1H-pyrazole, except substituting thiophen-3-ylboronic acid, 3-(1-(benzyloxy)naphthalen-2-yl)-5-(thiophen-3-yl)-1H-pyrazole was obtained in 90% yield: LCMS (ESI) R_{T}=1.301 min, m/z=383.3 [M+H]⁺.

2-(5-(Thiophen-3-yl)-1H-pyrazol-3-yl)naphthalen-1-ol: Using a procedure similar to that used in the preparation of 2-(5-(furan-3-yl)-1H-pyrazol-3-yl)naphthalen-1-ol '9133, except starting with 3-(1-(benzyloxy)naphthalen-2-yl)-5-(thiophen-3-yl)-1H-pyrazole, '9132 was obtained in 23% yield. 2-(5-(thiophen-3-yl)-1H-pyrazol-3-yl)naphthalen-1-ol. (23%). ¹H NMR (400 MHz, CDCl₃) δ (ppm) 8.46-8.43 (m, 1H), 7.82-7.80 (m, 1H) 7.72 (d, J=8.6 Hz, 1H), 7.60-7.59 (m, 1H), 7.54-7.50 (m, 3H), 7.44 (d, J=8.6 Hz, 1H), 7.40 (dd, J=5.1, 1.3 Hz, 1H), 6.93 (s, 1H); LCMS (ESI) R_{T}=1.255 min, m/z=293.1 [M+H]^{+.}

### Example 14: Procedural Details of Synthesis of '9134: 2-(5-(Benzofuran-2-yl)-1H-pyrazol-3-yl)naphthalen-1-ol

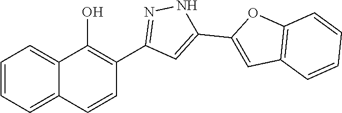 [SMILES: Oc1c(-c2cc(-c3cc4ccccc4o3)[nH]n2)ccc2ccccc12]

Using a two-step procedure similar to that used in the preparation of 2-(5-(furan-3-yl)-1H-pyrazol-3-yl)naphthalen-1-ol '9133, except starting with benzofuran-2-ylboronic acid, '9134 was obtained. ¹H NMR (400 MHz, CDCl₃) δ (ppm) 8.48-8.44 (m, 1H), 7.84-7.80 (m. 1H), 7.73 (d, J=8.6 Hz, 1H), 7.68 (d, J=7.2 Hz, 1H), 7.58 (d, J=8.0 Hz, 1H), 7.55-7.51 (m, 2H), 7.46 (d, J=8.6 Hz, 1H), 7.40 (t, J=7.7 Hz, 1H), 7.32 (t, J=7.3 Hz, 1H), 7.13 (d, J=3.3 Hz, 2H). LCMS (ESI) R_{T}=1.359 min, m/z=327.3 [M+H]⁺.

### Example 15: Procedural Details of Synthesis of '9130: 2-(5-(Benzo[b]thiophen-2-yl)-1H-pyrazol-3-yl)naphthalen-1-ol

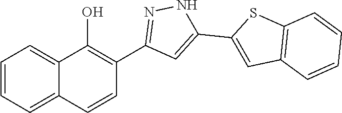 [SMILES: Oc1c(-c2cc(-c3cc4ccccc4s3)[nH]n2)ccc2ccccc12]

Using a two-step procedure similar to that used in the preparation of 2-(5-(furan-3-yl)-1H-pyrazol-3-yl)naphthalen-1-ol '9133, except starting with benzo[b]thiophen-2-ylboronic acid, '9130 was obtained. ¹H NMR (400 MHz, CDCl₃) δ (ppm) 8.48-8.40 (m, 1H), 7.95-7.86 (m, 2H), 7.84-7.80 (m, 1H), 7.73 (d, J=8.7 Hz, 1H), 7.61 (s, 1H), 7.55-7.51 (m. 2H), 7.49-7.42 (m, 3H), 7.06 (s, 1H). LCMS (ESI) R_{T}=1.439 min, m/z=343.3 [M+H]⁺.

### Example 16: Procedural Details of Synthesis of '9131: 2-(5-(Pyridin-4-yl)-1H-pyrazol-3-Anaphthalen-1-ol

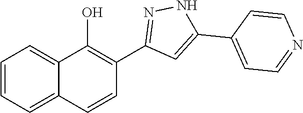 [SMILES: Oc1c(-c2cc(-c3ccncc3)[nH]n2)ccc2ccccc12]

Using a two-step procedure similar to that used in the preparation of 2-(5-(furan-3-yl)-1H-pyrazol-3-yl)naphthalen-1-ol '9133, except starting with pyridin-4-ylboronic acid, '9131 was obtained. ¹H NMR (400 MHz, MeOD) δ (ppm) 8.73 (d, J=5.6 Hz, 2H), 8.41 (d, J=8.1 Hz, 1H), 8.17-8.13 (m, 2H), 8.03 (bs, 1H), 7.90-7.83 (m, 1H), 7.68-7.54 (m, 4H). LCMS (ESI) R_{T}=0.854 min, m/z=288.2 [M+H]⁺.

### Example 17: Procedural Details of Synthesis of '9128: 2-(5-Ethynyl-1H-pyrazol-3-yl)naphthalen-1-ol

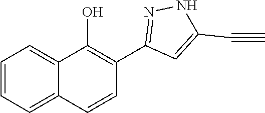 [SMILES: C#Cc1cc(-c2ccc3ccccc3c2O)n[nH]1]

Using a two-step procedure similar to that used in the preparation of 2-(5-(furan-3-yl)-1H-pyrazol-3-yl)naphthalen-1-ol '9133, '9128 was obtained (74%). ¹H NMR (400 MHz, MeOD) δ (ppm) 8.35-8.31 (m, 1H), 7.81-7.77 (m, 1H), 7.71 (d, J=8.7 Hz, 1H), 7.50-7.45 (m, 2H), 7.41 (d, J=8.7 Hz, 1H), 7.04 (s, 1H), 4.01 (bs, 1H). R_{T}=1.114 min, m/z=235.3 [M+H]⁺.

### Example 18: Procedural Details of Synthesis of '9769: 2-(5-(2-Methylthiophen-3-yl)-1H-pyrazol-3-yl)naphthalen-1-ol

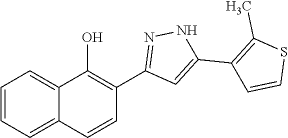 [SMILES: Cc1sccc1-c1cc(-c2ccc3ccccc3c2O)n[nH]1]

3-(1-(Benzyloxy)naphthalen-2-yl)-5-(2-methylthiophen-3-yl)-1H-pyrazole. To a solution containing 3-(1-(benzyloxy)naphthalen-2-yl)-5-bromo-1H-pyrazole (75 mg, 0.20 mmol) and THF (5.0 mL) was added (2-methylthiophen-3-yl)boronic acid (28.7 mg, 0.30 mmol), followed by bis(tri-tert-butylphosphine)-palladium(0) (10.1 mg, 0.020 mmol). The reaction mixture was degassed for 15 min and Cs₂CO₃ (aq, 1.0 M) (0.4 mL) was added. The reaction mixture was heated at 95° C. for 16 h, then cooled to rt and adsorbed on celite. The crude product was purified by flash chromatography using EtOAc/Hexane (0-90%) as eluents to afford 3-(1-(benzyloxy)naphthalen-2-yl)-5-(2-methylthiophen-3-yl)-1H-pyrazole (92.7 mg, 88%). R_{T}=1.323 min, m/z=397.3 [M+H]⁺.

2-(5-(2-Methylthiophen-3-yl)-1H-pyrazol-3-yl)naphthalen-1-ol. To a solution containing 3-(1-(benzyloxy)naphthalen-2-yl)-5-(2-methylthiophen-3-yl)-1H-pyrazole (90 mg, 0.23 mmol) in DCM (5.0 mL) at 0° C. was added a solution of BCl₃ (1.0 M in DCM) (0.5 mL, 0.5 mmol). The reaction mixture was allowed to slowly warm to rt and stir for 16 h. The reaction mixture was then washed with H₂O and NaHCO₃ (aq). The organic layer was dried with MgSO₄ and concentrated under reduced pressure. The crude product was purified by HPLC using 1% TFA (Aq)/MeCN as eluents to afford 2-(5-(2-methylthiophen-3-yl)-1H-pyrazol-3-yl)naphthalen-1-ol (14.6 mg, 21%). ¹H NMR (400 MHz, Acetone-D₆) δ (ppm) 8.39-8.36 (m, 1H), 7.93 (d, J=8.6 Hz, 1H), 7.86-7.84 (m, 1H) 7.53-7.49(m, 2H), 7.47 (d, J=8.6 Hz, 1H), 7.42 (q, J=5.3, 18.6 Hz, 1H), 7.17 (s, 1H), 2.73 (3s, 3H); LCMS (ESI) R_{T}=1.274 min, m/z=307.2 [M+H]⁺.

### Example 19: Procedural Details of Synthesis of '9952: -(5-(5-(Hydroxymethyl)thiophen-2-yl)-1H-pyrazol-3-yl)naphthalen-1-ol

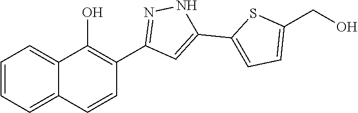 [SMILES: OCc1ccc(-c2cc(-c3ccc4ccccc4c3O)n[nH]2)s1]

(4-(3-(1-(Benzyloxy)naphthalen-2-yl)-1H-pyrazol-5-yl)thiophen-2-yl)methanol. Using a procedure similar to that used in the preparation of 3-(1-(Benzyloxy)naphthalen-2-yl)-5-(2-methylthiophen-3-yl)-1H-pyrazole, except using (5-(hydroxymethyl)thiophen-2-yl)boronic acid, (4-(3-(1-(benzyloxy)naphthalen-2-yl)-1H-pyrazol-5-yl)thiophen-2-yl)methanol was obtained (62.6 mg, 77%). LCMS (ESI) R_{T}=1.143 min, m/z=413.2 [M+H]⁺.

2-(5-(5-(Hydroxymethyl)thiophen-2-yl)-1H-pyrazol-3-yl)naphthalen-1-ol. Using a procedure similar to that used in the preparation of 2-(5-(2-methylthiophen-3-yl)-1H-pyrazol-3-yl)naphthalen-1-ol '9769, 2-(5-(5-(hydroxymethypthiophen-3-yl)-1H-pyrazol-3-yl)naphthalen-1-ol '9952 was obtained in 9% yield. ¹H NMR (400 MHz, Acetone-D₆) δ (ppm) 8.38-8.35 (m, 1H), 7.88-7.84 (m, 3H), 7.52 (m, 3H), 7.47 (d, J=8.6 Hz, 1H), 7.25 (s, 1H), 4.86 (d, J=5.0 Hz, 2H); LCMS (ESI) R_{T}=1.078 min, m/z=323.2 [M+H]⁺.

### Example 20: Procedural Details of Synthesis of '9767: 2-(5-(3-Methylpyridin-4-yl)-1H-pyrazol-3-yl)naphthalen-1-ol

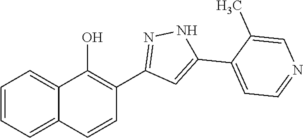 [SMILES: Cc1cnccc1-c1cc(-c2ccc3ccccc3c2O)n[nH]1]

Using a two-step procedure similar to that used in the preparation of 2-(5-(2-methylthiophen-3-yl)-1H-pyrazol-3-yl)naphthalen-1-ol '9769, except starting with (3-methylpyridin-4-yl)boronic acid, 2-(5-(3-methylpyridin-4-yl)-1H-pyrazol-3-yl)naphthalen-1-ol '9767 was obtained. ¹H NMR (400 MHz, MeOD) δ (ppm) 8.73 (s, 1H), 8.66 (d, J=6.0 Hz, 1H), 8.34 (dd, J=3.4, 6.1 Hz, 1H) 7.86-7.81 (m, 2H), 7.55-7.48 (m, 3H), 7.42 (s, 1H), 2.78 (s, 3H). LCMS (ESI) R_{T}=0.905 min, m/z=302.3 [M+H]⁺.

### Example 21: Procedural Details of Synthesis of '9768: (E)-2-(5-(Prop-1-en-1-yl)-1H-pyrazol-3-yl)naphthalen-1-ol

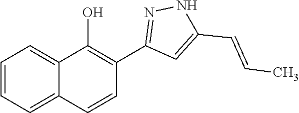 [SMILES: C/C=C/c1cc(-c2ccc3ccccc3c2O)n[nH]1]

Using a two-step procedure similar to that used in the preparation of 2-(5-(2-methylthiophen-3-yl)-1H-pyrazol-3-yl)naphthalen-1-ol '9769, except starting with 4,4,5,5-tetramethyl-2-[(1E)-prop-1-en-1-yl]-1,3,2-dioxaborolane, '9768 was obtained. ¹H NMR (400 MHz, Acetone-D₆) δ (ppm) 8.36-8.32 (m, 1H), 7.85-7.81 (m, 1H), 7.80 (d, J=8.6 Hz, 1H), 7.52-7.47 (m, 2H), 7.44 (d, J=8.9 Hz, 1H), 6.95 (s, 1H), 6.54-6.51 (m, m, 2H), 1.95-1.93 (m, 3H). LCMS (ESI): R_{T}=1.197 min, m/z=251.2 [M+H]⁺.

### Example 22: Procedural Details of Synthesis of '9625: 2-(5-(5-Methylthiophen-2-yl)-1H-pyrazol-3-yl)naphthalen-1-ol

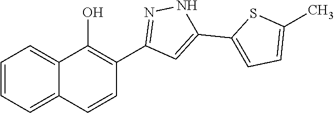 [SMILES: Cc1ccc(-c2cc(-c3ccc4ccccc4c3O)n[nH]2)s1]

2-Bromo-1-methoxynaphthalene. To a solution containing 2-bromonaphthalen-1-ol (2.0 g, 8.9 mmol) and 30 mL of MeCN was added KOH (1.0 g, 17.93 mmol). The reaction mixture was purged with Ar and methyl iodide (2.2 mL, 35.8 mmol) was added over 10 minutes. The reaction mixture was allowed to stir at rt for 16 h, diluted with H₂O (30 mL), and extracted with EtOAc (3×, 30 mL). The combined organic layers were washed with brine, dried over MgSO₄, and concentrated under reduced pressure. The crude product was purified by flash chromatography using EtOAc/Hexane (0-40%) as eluents to afford 1-(benzyloxy)-2-bromonaphthalene (1.62 g, 73%). ¹H NMR (400 MHz, CDCl₃) δ (ppm) 8.17 (d, J=7.8 Hz, 1H), 7.86 (d, J=7.8 Hz, 1H) 7.63-7.51 (m, 4H), 4.05 (s, 3H).

3-(1-Methoxynaphthalen-2-yl)-1H-pyrazole. Using a palladium-based procedure similar to that used with 3-(1-(benzyloxy)naphthalen-2-yl)-1H-pyrazole, 3-(1-methoxynaphthalen-2-yl)-1H-pyrazzole was prepared. (3.69 g, 93%). ¹H NMR (400 MHz, CDCl₃) δ (ppm) 8.22 (d, J=8.2 Hz, 1H), 8.18 (d, J=8.7 Hz, 1H), 7.86 (d, J=7.3Hz, 1H), 7.73 (d, J=2.4 Hz, 1H), 7.67 (d, J=8.7 Hz, 1H), 7.57-7.7.48 (m, 2H), 7.08 (d, J=2.4 Hz, 1H), 3.87 (s, 3H); LCMS (ESI) R_{T}=0.951 min, m/z=225.3 [M+H]⁺.

4,5-Dibromo-3-(1-methoxynaphthalen-2-yl)-1H-pyrazole. Using a bromination procedure similar to that used with 3-(1-(benzyloxy)naphthalen-2-yl)-4,5-dibromo-1H-pyrazole, 4,5-dibromo-3-(1-methoxynaphthalen-2-yl)-1H-pyrazole was prepared (59%). ¹H NMR (400 MHz, CDCl₃) δ (ppm) 8.20-8.13 (dd, J=8.3, 11.9 Hz, 2H), 8.06 (s, 1H), 8.04 (s, 1H), 7.73-7.62 (m, 2H). 3.76 (s, 3H).

5-Bromo-3-(1-methoxynaphthalen-2-yl)-1H-pyrazole. Using a lithiation procedure similar to that used with 3-(1-(benzyloxy)naphthalen-2-yl)-5-bromo-1H-pyrazole, 5-bromo-3-(1-methoxynaphthalen-2-yl)-1H-pyrazole was prepared (54%). ¹H NMR (400 MHz, MeOD) δ (ppm) 8.15 (d, J=8.1 Hz, 1H), 7.87 (d, J=7.4 Hz, 1H) 7.70 (d, J=8.6 Hz, 1H), 7.61-7.52 (m, 3H), 6.71 (s, 1H), 3.93 (s, 3H). LCMS (ESI) R_{T}=1.059 min, m/z=303.0 [M+H]⁺.

Regioisomer 4-bromo-3-(1-methoxynaphthalen-2-yl)-1H-pyrazole was isolated as a minor product (23%). ¹H NMR (400 MHz, MeOD) δ (ppm) 8.22-8.18 (m, 1H), 7.98 (d, J=8.4 Hz, 1H) 7.90-7.86 (m, 1H), 7.72 (d, J=8.7 Hz, 1H), 7.70 (s, 1H), 7.59-7.54 (m, 2H), 3.75 (s, 3H). LCMS (ESI) R_{T}=1.009 min, m/z=303.0 [M+H]⁺.

5-(1-Methoxynaphthalen-2-yl)-3-(5-methylthiophen-2-yl)-1H-pyrazole. To a solution containing 5-bromo-3-(1-methoxynaphthalen-2-yl)-1H-pyrazole (100 mg, 0.33 mmol) and THF (4.0 mL) was added (5-methylthiophen-2-yl)boronic acid (51.5 mg, 0.36 mmol) followed by bis(tri-tert-butylphosphine)-palladium(0) (8.5 mg, 0.016 mmol). The reaction mixture was degassed and Cs₂CO₃ (aq, 1.0 M) (0.7 mL) was added. The reaction mixture was heated at 95° C. for 16 h, then cooled to rt and adsorbed onto celite. The crude product was purified by flash chromatography using EtOAc/Hexane (0-90%) as eluents to afford 5-(1-methoxynaphthalen-2-yl)-3-(5-methylthiophen-2-yl)-1H-pyrazole (50.2 mg, 47%).LCMS (ESI) R_{T}=1.218 min, m/z=321.3 [M+H]⁺.

2-(5-(5-Methylthiophen-2-yl)-1H-pyrazol-3-yl)naphthalen-1-ol. To a solution containing 3-(1-methoxynaphthalen-2-yl)-5-(5-methylthiophen-2-yl)-1H-pyrazole (50 mg, 0.156 mmol) and DCE (10 mL) was added a solution of BBr₃ (1.0 M in DCM) (0.8 mL, 0.8mmol). The reaction mixture was heated at 58° C. for 16 h, then allowed to cool to rt. To the reaction mixture was added MeOH (5.0 mL) and the solvents were removed under reduced pressure. The crude product was purified by HPLC using 1% TFA (Aq)/MeCN as an eluent to afford 2-(5-(5-methylthiophen-2-yl)-1H-pyrazol-3-yl)naphthalen-1-ol ('9625) (12.4 mg, 26%). ¹H NMR (400 MHz, MeOD) δ (ppm) 8.32-8.32 (m, 1H), 7.90-7.78 (m, 1H) 7.75 (d, J=8.6 Hz, 1H), 7.49-7.45 (m, 2H), 7.41 (d, J=8.6 Hz, 1H), 7.28 (d, J=3.6 Hz, 1H), 6.93 (s, 1H), 6.83 (dd, J=3.4, 0.6 Hz, 1H), 2.55 (s, 3H); LCMS (ESI) R_{T}=1.376 min, m/z=307.1 [M+H]⁺.

### Example 23: Procedural Details of Synthesis of '9626: 2-(5-(Thiophen-2-yl)-1H-pyrazol-3-yl)naphthalen-1-ol

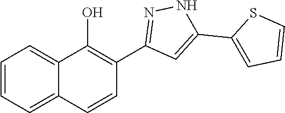 [SMILES: Oc1c(-c2cc(-c3cccs3)[nH]n2)ccc2ccccc12]

Using a procedure similar to that used in the preparation of 5-(1-methoxynaphthalen-2-yl)-3-(5-methylthiophen-2-yl)-1H-pyrazole, except using thiophen-2-ylboronic acid, 2-(5-(thiophen-2-yl)-1H-pyrazol-3-yl)naphthalen-1-ol was obtained in 81% yield. LCMS (ESI): R_{T}=1.158 min, m/z=307.1 [M+H]⁺.

Using a procedure similar to that used in the preparation of '9625, 2-(5-(thiophen-2-yl)-1H-pyrazol-3-yl)naphthalen-1-ol ('9626) was obtained in 31% yield. ¹H NMR (400 MHz, MeOD) δ (ppm) 8.35-8.33 (m, 1H), 7.81-7.77 (m, 2H) 7.54-7.51 (m, 2H), 7.49-7.46 (m, 2H), 7.41 (d, J=8.5 Hz, 1H), 7.18 (t, J=4.3 Hz, 1H), 7.03 (s, 1H); LCMS (ESI) R_{T}=1.287 min, m/z=293.2 [M+H]⁺.

### Example 24: Procedural Details of Synthesis of '6700: 2-(5-(1-Methyl-1H-indazol-5-yl)-1H-pyrazol-3-Anaphthalen-1-ol

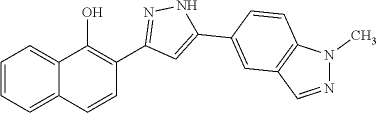 [SMILES: Cn1ncc2cc(-c3cc(-c4ccc5ccccc5c4O)n[nH]3)ccc21]

Using a procedure similar to that used in the preparation of 5-(1-methoxynaphthalen-2-yl)-3-(5-methylthiophen-2-yl)-1H-pyrazole, except using (1-methyl-1H-indazol-5-yl)boronic acid, 5-(3-(1-methoxynaphthalen-2-yl)-1H-pyrazol-5-yl)-1-methyl-1H-indazole was obtained in 46% yield. LCMS (ESI): R_{T}=1.093 min, m/z=355.0 [M+]⁺.

Using a procedure similar to that used in the preparation of '9625, 2-(5-(1-methyl-1H-indazol-5-yl)-1H-pyrazol-3-yl)naphthalen-1-ol ('6700) was obtained. ¹H NMR (400 MHz, MeOD) δ (ppm) 8.39-8.35 (m, 2H), 8.19 (s, 1H), 7.95 (dd, J=1.1, 8.9 Hz, 1H), 7.92-7.89 (m, 1H), 7.85 (d, J=8.7 Hz, 1H), 7.78 (d, J=8.7 Hz, 1H), 7.62-7.57 (m, 4H), 4.15 (s, 3H). LCMS (ESI) R_{T}=1.432 min, m/z=1.432 [M+H]⁺.

### Example 25: Procedural Details of Synthesis of '9624: 2-(5-(5-Methylfuran-2-yl)-1H-pyrazol-3-yl)naphthalen-1-ol

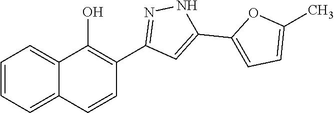 [SMILES: Cc1ccc(-c2cc(-c3ccc4ccccc4c3O)n[nH]2)o1]

Using a two-step procedure similar to that used in the preparation of 2-(5-(5-methylthiophen-2-yl)-1H-pyrazol-3-yl)naphthalen-1-ol '9625, except starting with (5-methylfuran-2-yl)boronic acid, '9624 was obtained. ¹H NMR (400 MHz, MeOD) δ (ppm) 8.35-8.30 (m, 1H), 7.81-7.75 (m, 2H), 7.49-7.45 (m, 2H), 7.42 (d, J=8.6 Hz, 1 H), 6.99 (s, 1H), 6.73 (d, J=3.2 Hz, 1H), 6.20 (d, J=3.2 Hz, 1H), 2.42 (s, 3H): LCMS (ESI) R_{T}=1.287 min, m/z=293.2 [M+H]⁺.

### Example 26: Procedural Details of Synthesis of '0071: 2-(5-(Oxazol-5-yl)-1H-pyrazol-3-yl)naphthalen-1-ol

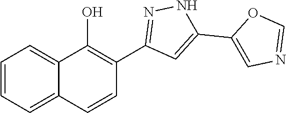 [SMILES: Oc1c(-c2cc(-c3cnco3)[nH]n2)ccc2ccccc12]

Using a two-step procedure similar to that used in the preparation of 2-(5-(5-methylthiophen-2-yl)-1H-pyrazol-3-yl)naphthalen-1-ol '9625, except starting with oxazol-5-ylboronic acid, '0071 was obtained. ¹H NMR (400 MHz, MeOD) δ (ppm) 8.37-8.33 (m, 2H), 7.83-7.77 (m, 2 H), 7.59 (s, 1H), 7.51-7.47 (m, 2H), 7.45 (d, J=8.67 Hz, 1H), 7.21 (s, 1H): LCMS (ESI) R_{T}=1.081 min, m/z=278.2 [M+H]⁺.

### Example 27: Procedural Details of Synthesis of '9627: 2-(5-(4-Methylthiophen-2-yl)-1H-pyrazol-3-yl)naphthalen-1-ol

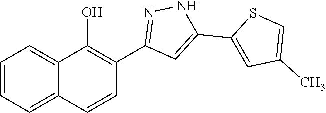 [SMILES: Cc1csc(-c2cc(-c3ccc4ccccc4c3O)n[nH]2)c1]

Using a two-step procedure similar to that used in the preparation of 2-(5-(5-methylthiophen-2-yl)-1H-pyrazol-3-yl)naphthalen-1-ol '9625, except starting with (4-methylthiophen-2-yl)boronic acid, '9627 was obtained. ¹H NMR (400 MHz, MeOD) δ (ppm) 8.36-8.30 (m, 1H), 7.82-7.76 (m, 2H), 7.50-7.41 (m, 3H), 7.32 (s, 1H), 7.09 (s, 1H), 6.99 (s, 1H), 2.33 (s, 3H). LCMS (ESI) R_{T}=1.348 min, m/z=307.1 [M+H]⁺.

### Example 28: Procedural Details of Synthesis of '8233: 2-(2′-Methyl-2H,2′H-[3,3′-bipyrazol]-5-yl)naphthalen-1-ol

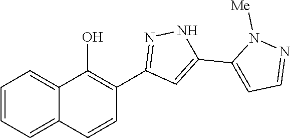 [SMILES: Cn1nccc1-c1cc(-c2ccc3ccccc3c2O)n[nH]1]

Using a two-step procedure similar to that used in the preparation of 2-(5-(5-methylthiophen-2-yl)-1H-pyrazol-3-yl)naphthalen-1-ol '9625, except starting with (1-methyl-1H-pyrazol-5-yl)boronic acid, '8233 was obtained. ¹H NMR (400 MHz, MeOD) δ (ppm) 8.38-8.36 (m, 1H), 7.87-778 (m, 2H), 7.66-7.42 (m, 4H), 7.17 (s, 1H), 6.69 (s, 1H), 4.10 (s, 3H). LCMS (ESI) R_{T}=1.063 min, m/z=291.3 [M+H]⁺.

### Example 29: Procedural Details of Synthesis of '6699: 2-(1-Methyl-1H,2′H-[3,3′-bipyrazol]-5′-yl)naphthalen-1-ol

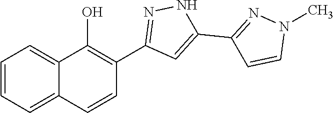 [SMILES: Cn1ccc(-c2cc(-c3ccc4ccccc4c3O)n[nH]2)n1]

Using a two-step procedure similar to that used in the preparation of 2-(5-(5-methylthiophen-2-yl)-1H-pyrazol-3-yl)naphthalen-1-ol '9625, except starting with (1-methyl-1H-pyrazol-3-yl)boronic acid, '6699 was obtained. ¹H NMR (400 MHz, MeOD) δ (ppm) 8.35-8.32 (m, 1H), 8.04 (s, 1H), 7.89 (S, 1H), 7.81-7.76 (m, 2H), 7.49-7.45 (m, 2H), 7.42 (d, J=8.4 Hz, 1H), 6.95 (s, 1H), 3.99 (s, 3H). LCMS (ESI) R_{T}=1.071 min , m/z=291.2 [M+H]⁺.

### Example 30: Procedural Details of Synthesis of '4083: 5-Fluoro-2-(5-(furan-2-yl)-1H-pyrazol-3-yl)phenol

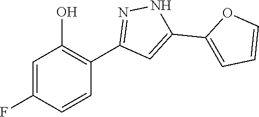 [SMILES: Oc1cc(F)ccc1-c1cc(-c2ccco2)[nH]n1]

3,5-Dibromo-1H-pyrazole. To a mixture containing 3,4,5-tribromo-1H-pyrazole (10.0 g, 32.8 mmol) and tetrahydrofuran (130 mL) at −78° C. under an atmosphere of argon was added n-butyllithium (28.8 mL, 72.1 mmol, 2.5 M in hexanes) over 20 minutes. The reaction mixture was stirred at −78° C. for 2 h, and then a solution of methanol/tetrahydrofuran (20 mL/30 mL) was added dropwise. The reaction mixture was allowed to warm to room temperature and concentrated under reduced pressure. The resulting material was diluted with diethyl ether (500 mL), washed with aqueous hydrochloric acid (1 N, 25 mL) and saturated aqueous sodium chloride solution (20 mL), dried over sodium sulfate, filtered, and concentrated under reduced pressure. The 3,5-dibromo-1H-pyrazole was obtained as a yellow solid (4.5 g, 60%). ¹H NMR (400 MHz, DMSO-d⁶): δ 13.88 (brs, 1H), 6.59 (s, 1H). LC-MS: R_{T} 2.27 min, m/z=224.84, 226.78, 228.80 [M+H]⁺ (two Br pattern).

3-Bromo-5-(furan-2-yl)-1H-pyrazole. To a 500 mL flask was added 3,5-dibromo-1H-pyrazole (11.3 g, 50 mmol), furan-2-ylboronic acid (5.6, 50 mmol), potassium carbonate (13.8 g, 100 mmol), 1,1′-Bis(diphenylphosphino)ferrocene palladium (II) chloride (2.04 g , 2.5 mmol), dioxane (200 mL), and water (5 mL). The reaction mixture was heated at 105° C. in an oil bath overnight. After cooling to room temperature, the reaction was filtered and the volatiles were removed. The residue was subjected to silica gel chromatography using ethyl acetate/hexane (5:1, v/v) as an eluent to obtain 4.8 g (45%) of 3-bromo-5-(furan-2-yl)-1H-pyrazole, ¹H-NMR (400 MHz, DMSO-d⁶): δ 13.67 (brs, 1H), 7.80 (s, 1H), 6.87 (d, J=3.2 Hz, 1H), 6.67-6.63(m, 2H). LC-MS: R_{T} 7.39 min, m/z=212.90, 214.92 [M+H]⁺ (one Br pattern).

5-Fluoro-2-(5-(furan-2-yl)-1H-pyrazol-3-yl)phenol. To a 15 mL pressure reaction vial was added (4-fluoro-2-hydroxyphenyl)boronic acid (234 mg, 1.5 mmol), 3-bromo-5-(furan-2-yl)-1H-pyrazole (213 mg, 1.0 mmol), potassium carbonate (276 mg, 2.0 mmol), 1,1′-bis(diphenylphosphino)ferrocene palladium (II) chloride (82 mg, 0.1 mmol), dioxane (2 mL), and water (0.2 mL). The reaction mixture was heated at 105° C. in an oil bath for 24 h. After cooling to room temperature, 10 mL of water was added, the pH was adjusted to 2-3, and the mixture was extracted with DCM (3×30 mL). The organic layers were collected and dried over magnesium sulfate, and the volatiles were removed. The residue was subjected to silica gel chromatography using 10% to 100% dichloromethane in hexane as an eluent, to obtain '4083 (100 mg, 41.0%). ¹H NMR (400 MHz, CDCl₃): δ 10.95 (brs, 1H), 10.16 (brs, 1H), 7.55-7.51 (m, 2H), 6.77-6.70 (m, 3H), 6.68-6.63 (m, 1H), 6.54 (dd, J=2.0 Hz, 1.6 Hz 1H,). LC-MS, R_{T}: 8.52 min, m/z=245.08 [M+H]⁺.

### Example 31: Procedural Details of Synthesis of '3918: 3-(5-(Furan-2-yl)-1H-pyrazol-3-yl)naphthalen-2-ol

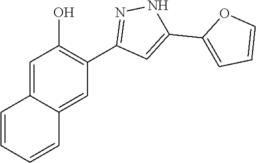 [SMILES: Oc1cc2ccccc2cc1-c1cc(-c2ccco2)[nH]n1]

To a 15 mL pressure reaction vial was added (3-hydroxynaphthalen-2-yl) boronic acid (226 mg, 1.2 m mol), 3-bromo-5-(furan-2-yl)-1H-pyrazole (213 mg, 1.0 mmol), potassium carbonate (276 mg, 2.0 mmol), 1,1′-Bis(diphenylphosphino)ferrocene palladium (II) chloride (82 mg, 0.1 mmol), dioxane (2 mL), water (0.2 mL). The reaction mixture was heated at 105° C. in an oil bath overnight. After cooling to room temperature, the reaction mixture was filtered, and the volatiles were removed under reduced pressure. The residue was purified by silica gel column using ethyl acetate/hexane (4:1, v/v) as an eluent, to obtain 100 mg (36%) of '3918. ¹H NMR (600 MHz, CD₃OD): δ 8.25 (s, 1H), 7.83 (d, J=5.2 Hz, 1H), 7.65 (d, 1H), 7.64 (s, 1H), 7.40-7.42 (m, 1H), 7.28-7.32 (m, 2H), 7.18 (s, 1H), 6.84 (s, 1H), 6.59 (s, 1H). LC-MS, R_{T} 9.03 min, m/z=277.22 [M+H]⁺.

### Example 32: Procedural Details of Synthesis of '4087: 3-(5-(Furan-2-yl)-1H-pyrazol-3-yl)naphthalen-2-ol

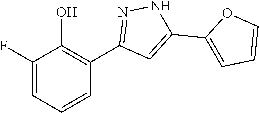 [SMILES: Oc1c(F)cccc1-c1cc(-c2ccco2)[nH]n1]

To a 15 mL pressure reaction vial was added (3-fluoro-2-hydroxyphenyl)boronic acid (234 mg, 1.5 mmol), 3-bromo-5-(furan-2-yl)-1H-pyrazole (213 mg, 1.0 mmol), potassium carbonate (276 mg, 2.0 mmol), 1,1′- Bis(diphenylphosphino)ferrocene palladium (II) chloride (82 mg, 0.1 mmol), dioxane (2 mL), and water (0.4 mL). The reaction mixture was heated at 105° C. in an oil bath for 24 h. After cooling to room temperature, 10 mL of water was added and the pH was adjusted to 2-3, and the mixture was extracted with DCM (3×30 mL). The organic layers were collected and dried over magnesium sulfate, and the volatiles were removed. The residue was subjected to silica gel chromatography using 50% to 100% dichloromethane in Hexane as an eluent, to provide 80 mg (33%) of '4087. ¹H NMR (400 MHz, CDCl₃): δ 10.68 (brs, 1H), 10.41 (brs, 1H), 7.52-7.51 (dd, J=1.2, 0.8 Hz, 1H), 7.38-7.35 (dt, J=7.6, 1.6 Hz, 1H,), 7.09-7.04 (m, 1H), 6.88-6.83 (m, 2H), 6.73-6.72 (dd, 1H), 6.55-6.54 (dd, J=2.0, 1.6 Hz, 1H). LC-MS: R_{T} 8.43 min, m/z=245.20 [M+H]⁺.

### Example 33: Procedural Details of Synthesis of '4082: 4-Fluoro-2-(5-(furan-2-yl)-1H-pyrazol-3-yl)phenol

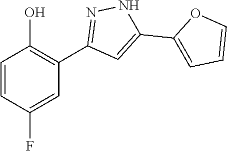 [SMILES: Oc1ccc(F)cc1-c1cc(-c2ccco2)[nH]n1]

To a 15 mL pressure reaction vial was added (5-fluoro-2-hydroxyphenyl)boronic acid (234 mg, 1.5 mmol), 3-bromo-5-(furan-2-yl)-1H-pyrazole (213 mg, 1.0 mmol), potassium carbonate (276 mg, 2.0 mmol), 1,1′-is(diphenylphosphino)ferrocene palladium (II) chloride (82 mg, 0.1 mmol), dioxane (2 mL), and water (0.2 mL). The reaction mixture was heated to 105° C. in an oil bath for 24 h. After cooling to room temperature, 10 mL of water was added, the pH was adjusted to 2-3, and the mixture was extracted with DCM (3×30 mL). The organic layers were collected and dried over magnesium sulfate, and the volatiles were removed. The residue was subjected to silica gel chromatography using 10% to 100% dichloromethane in Hexane as an eluent to obtain 100 mg (54%) of '4082. ¹H NMR (400 MHz, CDCl₃): δ 10.46 (brs, 1H), 10.31 (brs, 1H), 7.51 (d, J=1.6 Hz, 1H), 7.29-7.26 (dd, J=2.0, 0.8 Hz, 1H), 6.97-6.94 (m, 2H), 6.79 (s, 1H), 6.72 (d, J=3.2 Hz, 1H), 6.54 (dd, J=5.0, 1.6 Hz, 1H). LC-MS: R_{T} 8.41 min, m/z=245.23 [M+H]⁺.

### Example 34: Procedural Details of Synthesis of '7268: 3-(5-(Furan-2-yl)-1H-pyrazol-3-yl)pyridin-2-ol

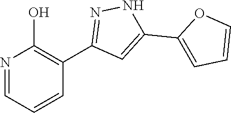 [SMILES: Oc1ncccc1-c1cc(-c2ccco2)[nH]n1]

To a 15 mL pressure reaction vial was added (2-fluoropyridin-3-yl)boronic acid (211 mg, 1.5 mmol), 3-bromo-5-(furan-2-yl)-1H-pyrazole (213 mg, 1.0 mmol), potassium carbonate (276 mg, 2.0 mmol), 1,1′-bis(diphenylphosphino)ferrocene palladium (II) chloride (82 mg, 0.1 mmol), dioxane (3 mL), and water (0.2 mL). The reaction mixture was heated at 80° C. in an oil bath overnight. After cooling to room temperature, the reaction mixture was filtered and the volatiles were removed. The residue was subjected to silica gel chromatography using ethyl acetate/hexane (4:1, v/v) as an eluent to obtain 178 mg (77.7%) of 2-fluoro-3-(5-(furan-2-yl)-1H-pyrazol-3-yl)pyridine. LC-MS: R_{T}: 7.48 min, m/z=229.98 [M+H]⁺.

A mixture containing 178 mg of 2-fluoro-3-(3-(furan-2-yl)-1H-pyrazol-5-yl)pyridine, 7 mL of acetic acid, and 3 mL of water was heated at 100° C. overnight. The solvent was removed under reduced pressure and the residue was purified by silica gel chromatography using 5% methanol in DCM as an eluent, to give 150 mg (85%) of '7268. ¹H NMR (400 MHz, CD₃OD): δ 8.16-8.13 (dd, 1H, J=5.2, 2.0 Hz), 7.58-7.57 (dd, J=1.2, 0.8 Hz, 1H), 7.51-7.49 (dd, J=4.4, 2 Hz, 1H), 7.10 (s, 1H), 6.77-6.76 (dd, J=2.8, 0.4 Hz, 1H), 6.56-6.53 (m, 2H). LC-MS: R_{T} 6.67 min, m/z=228.00 [M+H]⁺.

### Example 35: Procedural Details of Synthesis of '3989: 3-(5-(Furan-2-yl)-1H-pyrazol-3-yl)pyridin-2-amine

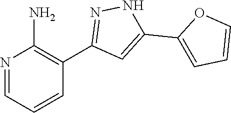 [SMILES: Nc1ncccc1-c1cc(-c2ccco2)[nH]n1]

To a 15 mL pressure reaction vial was added 3-(4,4,5,5-tetramethyl-1,3,2-dioxaborolan-2-yl)pyridin-2-amine (95%, 330 mg, 1.5 mmol), 3-bromo-5-(furan-2-yl)-1H-pyrazole (213 mg, 1.0 mmol), potassium carbonate (276 mg, 2.0 mmol), 1,1′-bis(diphenylphosphino)ferrocene palladium (II) chloride (82 mg, 0.1 mmol), dioxane (2 mL), and water (0.2 mL). The reaction mixture was heated at 105° C. in an oil bath overnight. After cooling to room temperature, the reaction mixture was filtered, and the volatiles were removed under reduced pressure. The residue was purified by silica gel chromatography using 50%-70% ethyl acetate in hexane as an eluent, to obtain 157 mg (69%) of '3989. ¹H NMR (400 MHz, CDCl₃, 55° C.): δ 11.33 (brs, 1H), 8.10-8.09 (dd, J=3.2, 1.6 Hz, 1H,), 7.80-7.78 (dd, J=6.0, 1.6 Hz, 1H), 7.50-7.49 (dd, J=1.2, 0.8 Hz, 1H,), 6.81 (s, 1H), 6.71-6.66 (m, 2H), 6.52-6.51 (dd, J=1.6, 1.6 Hz, 1H), 6.46 (brs, 2H). LC-MS: R_{T} 6.03 min, m/z=227.05 [M+H]⁺.

### Example 36: Procedural Details of Synthesis of '7092: 2-(5-(Furan-2-yl)-1H-pyrazol-3-yl)pyridine

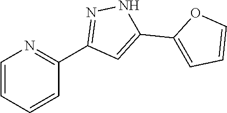 [SMILES: c1ccc(-c2cc(-c3ccco3)[nH]n2)nc1]

Prepared by a similar procedure as used for the preparation of '4083,except using pyridin-2-ylboronic acid in the last step. 1H NMR (600 MHz, CDCl₃): δ 11.60 (s, 1H), 8.9 (dd, J=6.0, 1.2 Hz, 1H), 8.80 (td, J=7.8, 1.2 Hz, 1H), 8.55 (d, J=7.8 Hz, 1H), 7.65 (d, J=1.8 Hz, 1H), 7.44 (s, 1H), 7.08 (d, J=1.8 Hz, 1H), 6.67 (dd, J=3.6, 1.8 Hz, 1H). LC-MS: R_{T} 5.55 min, m/z=212.30 [M+H]⁺.

### Example 37: Procedural Details of Synthesis of '0485: 2-(5-(Furan-2-yl)-1H-1,2,4-triazol-3-yl)naphthalen-1-ol

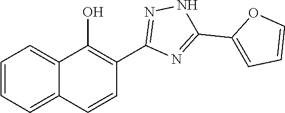 [SMILES: Oc1c(-c2n[nH]c(-c3ccco3)n2)ccc2ccccc12]

Furan-2-carboximidamide hydrochloride. To a solution containing sodium methoxide (0.11 g, 2.0 mmol) and methanol (20 mL) was added 2-cyanofuran (1.9 g, 20 mmol). The reaction mixture was allowed to stir at room temperature for 2 h. Ammonium chloride (1.2 g, 23 mmol) was added and the reaction mixture was stirred at room temperature for a further 3 h. The white precipitate was filtered, washed with ether and dried in vacuo to provide 2.5 g (17 mmol, 85% yield) of furan-2-carboximidamide hydrochloride as a white solid that was used with no further purification.

1-Hydroxy-2-naphthohydrazide. Methyl 1-hydroxy-2-naphthoate (1.0 g, 4.9 mmol) was suspended in EtOH (10 mL) and treated with hydrazine monohydrate (0.75 mL, 10 mmol). The reaction mixture was heated at 90° C. for 16 h, allowed to cool to room temperature, and filtered. The off-white crystals obtained were washed with MeOH and dried in vacuo to provide 0.59 g (2.9 mmol, 59% yield) of 1-hydroxy-2-naphthohydrazide as off-white crystals. ¹H NMR (600 MHz, DMSO): δ 14.41 (br, 1H), 10.29 (br, 1H), 8.25 (d, J=7.8 Hz, 1H), 7.85 (d, J=7.8 Hz, 1H), 7.82 (d, J=9.0 Hz, 1H), 7.62 (dt, J=7.2, 1.2 Hz, 1H), 7.54 (dt, J=8.4, 1.2 Hz, 1H), 7.33 (d, J=9.0 Hz, 1H), 4.73 (br, 2H).

2-(5-(Furan-2-yl)-1H-1,2,4-triazol-3-yl)naphthalen-1-ol. To a solution containing sodium methoxide (0.11 g, 2.0 mmol) and ethanol (10 mL) was added furan-2-carboximidamide hydrochloride (0.22 g, 1.5 mmol). The reaction mixture was stirred at rt for 10 minutes. The slurry was filtered and the filtrate was transferred to a microwave vial. 1-Hydroxy-2-naphthohydrazide (0.20 g, 1.0 mmol) was added to the reaction solution and the mixture was heated at 160° C. for 1 h. The reaction solution was allowed to cool to room temperature, acidified with 1 M HCl in Et₂O, and purified by column chromatography using 25-50% EtOAc/hexanes as eluent. The product thus obtained was washed with MeOH and dried in vacuo to afford 47 mg (0.17 mmol, 17% yield) of '0485 as a tan solid. ¹H NMR (600 MHz, DMSO, 80° C.): δ 14.77 (brs, 1H), 12.40 (brs, 1H), 8.34 (d, 1H, J=12.6 Hz), 8.04 (d, 1H, J=12.6 Hz), 7.89 (d, 2H, J=10.8 Hz), 7.58 (m, 3H), 7.18 (brs, 1H), 6.72 (s, 1H); ¹³C NMR (150 MHz, DMSO, 80° C.) δ 152.71, 144.18, 134.46, 127.31, 127.16, 125.37, 124.32, 122.28, 118.70, 111.53. MS (ESI) for C₁₆H₁₁N₃O₂ calc 277.09, found 277.93 [M+H]⁺.

### Example 38: Procedural Details of Synthesis of '0394: 2-(5-(Furan-2-yl)-4-methyl-1H-pyrazol-3-yDphenol

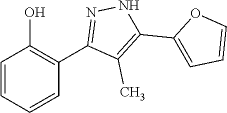 [SMILES: Cc1c(-c2ccccc2O)n[nH]c1-c1ccco1]

2-((tert-Butyldimethylsilyfloxy)benzaldehyde. A solution containing salicylaldehyde (0.5 mL, 5.0 mmol), triethylamine (0.84 mL, 6.0 mmol), and DMAP (12 mg, 0.1 mmol) in DCM (17 mL) was cooled to 0° C. A solution of TBSCl (0.9 g, 6 mmol) in DCM (3 mL) was added dropwise and the reaction mixture was allowed to warm to room temperature. The reaction progress was monitored by TLC and, upon completion, the reaction was quenched with sat. aq. NaHCO₃ and washed with brine. The organic layer was dried over Na₂SO₄ and concentrated to give 2-((tert-butyldimethylsilyl)oxy)benzaldehyde as a white solid (1.18 g, 100% yield) that was used without further purification.

N′-(2-((tert-Butyldimethylsilyl)oxy)benzylidene)-4-methylbenzenesulfonohydrazide. A solution containing 2-((tert-butyldimethylsilypoxy)benzaldehyde (1.18 g, 5.0 mmol) in MeOH (25 mL) was treated with tosylhydrazide (1.0 g, 5.3 mmol). The reaction progress was monitored by TLC, and upon completion, the reaction suspension was cooled to 0° C. and filtered to give a white solid. The solid was washed with cold MeOH and dried in vacuo, to provide 1.5 g (75% yield) of N′-(2-((tert-butyldimethylsilyl)oxy)benzylidene)-4-methylbenzenesulfonohydrazide as a white solid. ¹H NMR (600 MHz, CDCl₃): δ 8.07 (s, 1H), 7.85 (d, J=8.4 Hz, 2H), 7.77 (dd, J=8.4, 1.8 Hz, 1H), 7.62 (s, 1H), 7.29 (d, J=8.4 Hz, 2H), 7.21 (dt, J=8.4, 1.8 Hz, 1H), 6.92 (t, J=7.8 Hz, 1H), 6.74 (d, J=8.4 Hz, 1H), 2.39 (s, 3H), 0.95 (s, 9H), 0.16 (s, 6H).

(E)-2-(2-Nitroprop-1-en-1-yl)furan. To a solution of nitroethane (13 mL, 181 mmol) in acetic acid (30 mL) was added butylamine (6.8 mL, 69 mmol) and furfural (5 mL, 60 mmol). The resulting mixture was heated at 80° C. for 2 h. The reaction progress was monitored by TLC and, upon completion, the reaction was allowed to cool to room temperature and treated with water while stirring vigorously. The crude product that precipitated was filtered, washed with water, and purified by column chromatography using 5-10% EtOAc/hexanes as an eluent, to provide 7.35 g (80% yield) of (E)-2-(2-nitroprop-1-en-1-yl)furan as yellow crystals. ¹H NMR (600 MHz, CDCl₃): δ 7.84 (s, 1H), 7.62 (d, J=1.8 Hz, 1H), 6.80 (d, J=3.6 Hz, 1H), 6.56 (dd, J=3.0, 1.8 Hz, 1H), 2.57 (s, 3H).

2-(5-(Furan-2-yl)-4-methyl-1H-pyrazol-3-yl)phenol. A suspension containing (E)-2-(2-nitroprop-1-en-1-yl)furan (0.40 g, 1.0 mmol), N-(2-((tert-butyldimethylsilyl)oxy)benzylidene)-4-methylbenzenesulfonohydrazide (0.31 g, 2.0 mmol), potassium carbonate (0.14 g, 1.0 mmol), and DABCO (67 mg, 0.60 mmol) in THF (10 mL, 0.1 M) was heated at 65° C. for 16 h. The reaction mixture was allowed to cool to room temperature and treated with a solution of TBAF (1.2 mL, 1.2 mmol) in THF. The reaction progress was monitored by TLC and, upon completion, the reaction was quenched with 1N HCl and extracted with EtOAc. The combined organic layers were washed with brine and dried over Na₂SO₄. The crude product was purified by normal phase column chromatography using 20-50% EtOAc/hexanes, followed by column chromatography using 0-5% MeOH/DCM, followed by reverse phase column chromatography using 0-100% CH₃CN/H₂O to provide 19 mg (7% yield) of '0394 as a white solid. ¹H NMR (600 MHz, CDCl₃): δ 10.56 (brs, 1H), 9.78 (brs, 1H), 7.51 (d, 1H, J=0.6 Hz), 7.17 (t, 1H, J=7.2 Hz), 7.10 (d, 1H, J=7.8 Hz), 6.99 (d, 1H, J=7.8 Hz), 6.70 (d, 1H, J=7.8 Hz), 6.49 (dd, 1H, J=3, 1.8 Hz), 6.33 (dd, 1H, J=3, 0.6 Hz), 2.34 (s, 3H); ¹³C NMR (150 MHz, CDCl₃): δ 142.63, 129.62, 127.79, 119.18, 117.14, 111.31, 110.02, 10.4. MS (ESI) for C₁₈H₁₄N₂O₂ calc. 240.09, found 241.08 [M+H]⁺.

### Example 39: Procedural Details of Synthesis of '1790: 2-(4,5-Dihydro-1H-furo[3,2-g]indazol-3-yDnaphthalen-1-ol

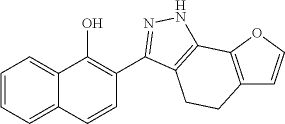 [SMILES: Oc1c(-c2n[nH]c3c2CCc2ccoc2-3)ccc2ccccc12]

Ethyl 4-(furan-3-yl)butanoate. Zn powder (1.96 g, 30 mmol) and LiCl (1.0 g, 24 mmol) were dried under vacuum, cooled to room temperature, and suspended in anhydrous THF (20 mL) under an atmosphere of nitrogen. 1,2-Dibromoethane (34 μL, 0.4 mmol) was added and the solution was heated at reflux until activation of the zinc was observed by vigorous effervescence. A solution of iodine (100 mg, 0.4 mmol) in THF (1 mL) was then added to the reaction mixture and the solution was heated until the red color had dissipated. Ethyl-4-bromobutyrate (3.9 g, 20 mmol) was added by syringe and the reaction mixture was heated at 55° C. for 16 h, then cooled to room temperature and allowed to settle. 3-bromofuran (1.79 g, 12.2 mmol) was weighed into a 100 mL RBF and dissolved in anhydrous THF (12 mL). XPhos-Pd-G3 (0.31 g, 0.37 mmol) was added and the reaction solution was degassed with nitrogen x3. The prepared organozinc reagent (20 mL) was transferred rapidly by syringe to the reaction solution and stirred at room temperature for 16 h. The reaction solution was evaporated onto Celite and purified by silica gel chromatography in 5% EtOAc/hexanes to provide 1.0 g (5.5 mmol, 46% yield) of ethyl 4-(furan-3-yl)butanoate as a clear colorless oil. ¹H NMR (600 MHz, CDCl₃): δ 7.33 (t, J=1.8 Hz, 1H), 7.20 (m, 1H), 6.25 (s, 1H), 4.10 (q, J=7.0 Hz, 2H), 2.44 (t, J=7.5 Hz, 2H), 2.30 (t, J=7.5 Hz, 2H), 1.87 (quint, J=7.5 Hz, 2H), 1.23 (t, J=7.2 Hz, 3H).

4-(Furan-3-yl)butanoic acid. A mixture containing ethyl 4-(furan-3-yl)butanoate (2.1 g, 11.7 mmol) and potassium hydroxide (17 mL, 34 mmol, 2 M in 95% EtOH/H₂O) was heated at 50° C. for 16 h. The reaction solution was cooled to room temperature and extracted with EtOAc. The organic layer was discarded and the aqueous layer was acidified with 1N HCl (35 mL) and extracted twice with EtOAc. The organic layer was dried over Na₂SO₄, filtered and concentrated to give 1.71 g (94% yield, 11.1 mmol) of 4-(furan-3-yl)butanoic acid as an orange oil that was used without further purification. ¹H NMR (600 MHz, CDCl₃): δ 7.33 (t, J=1.5 Hz, 1H), 7.21 (m, 1H), 6.25 (s, 1H), 2.47 (t, J=7.5 Hz, 2H), 2.37 (t, J=7.5 Hz, 2H), 1.88 (quint, J=7.4 Hz, 2H).

5,6-Dihydrobenzofuran-7(4H)-one. A mixture containing 4-(furan-3-yl)butanoic acid (1.71 g, 11.1 mmol) and DCM (22 mL) was treated with DMF (86 μL, 1.11 mmol). The reaction mixture was cooled to 0° C. and treated dropwise with oxalyl chloride (1.2 mL, 13.9 mmol). The ice bath was removed and the reaction solution was allowed to warm to room temperature and stirred for 1 h. The reaction solution was cooled back down to 0° C. and SnCl₄ (1.3 mL, 11.1 mmol) was added dropwise. The reaction progress was monitored by LCMS, and upon completion (0.5 h), the reaction solution was quenched with water and filtered through a pad of Celite with DCM. The filtrate was extracted twice with DCM and the combined organic layers were washed with 20 mL of 5% aqueous NaOH. The crude product was purified by silica gel chromatography using 25-40% EtOAc/hexanes as an eluent to provide 1.16 g (8.5 mmol, 77% yield) of 5,6-dihydrobenzofuran-7(4H)-one. ¹H NMR (600 MHz, CDCl₃): δ 7.54 (d, J=1.8 Hz, 1H), 6.39 (d, J=1.8 Hz, 1H), 2.75 (t, J=6.3 Hz, 2H), 2.55 (m, 2H), 2.14 (quint, J=6.3 Hz, 2H). MS (ESI) for C₈H₈O₂ calc. 136.05, found 136.88 [M+H]⁺.

(E)-6-((dimethylamino)methylene)-5,6-dihydrobenzofuran-7(4H)-one. A mixture containing 5,6-dihydrobenzofuran-7(4H)-one (1.16 g, 8.5 mmol) and DMFDMA (6.4 mL, 43 mmol) was heated at 200° C. for 0.5 h in a microwave reactor. The reaction mixture was loaded onto Celite and purified by silica gel chromatography in 100% EtOAc to provide 1.09 g (68% yield, 5.7 mmol) of E)-6-((dimethylamino)methylene)-5,6-dihydrobenzofuran-7(4H)-one. ¹H NMR. (400 MHz, CDCl₃): δ 7.47 (d, J=8.4 Hz, 2H), 6.32 (d, J=2.0 Hz, 1H), 3.07 (s, 6H), 2.96 (t, J=6.6 Hz, 2H), 2.66 (t, J=6.8 Hz, 2H).

4,5-Dihydro-2H-furo[3,2-g]indazole. A mixture containing (E)-6-((dimethylamino)methylene)-5,6-dihydrobenzofuran-7(4H)-one (1.25 g, 6.6 mmol) and EtOH (40 mL) was treated with hydrazine monohydrate (1.0 mL, 13 mmol) and heated at 60° C. for 16 h. The solvent was removed in vacuo and the crude product was dissolved in EtOAc and washed with water and brine ×2. The organic layer was dried over Na₂SO₄, filtered and concentrated to give 1.04 g (98% yield, 6.5 mmol) of 4,5-dihydro-2H-furo[3,2-g]indazole, which was used without further purification.

2-(Tetrahydro-2H-pyran-2-yl)-4,5-dihydro-2H-furo[3,2-g]indazole. A mixture containing 4,5-dihydro-2H-furo[3,2-g]indazole (1.04 g, 6.5 mmol) and THF (30 mL) was treated with DHP (1.2 mL, 13 mmol) and pyr·pTsOH (0.16 g, 0.65 mmol). The reaction mixture was heated at 65° C. for 16 h, cooled to room temperature, loaded directly onto Celite, and purified by silica gel chromatography in 10-20% EtOAc/hexanes to provide 0.96 g (60% yield, 3.9 mmol) of 2-(tetrahydro-2H-pyran-2-yl)-4,5-dihydro-2H-furo[3,2-g]indazole as a clear oil. ¹H NMR (600 MHz, CDCl₃): 7.35 (d, J=1.8 Hz, 1H), 7.31 (s, 1H), 6.31 (d, J=1.8 Hz, 1H), 5.32 (dd, J=12.0, 2.4 Hz, 1H), 4.04 (m, 1H), 3.67 (t, J=12.6 Hz, 1H), 2.76 (m, 4H), 2.03 (m, 2H), 1.65 (m, 2H), 1.58 (m, 2H). MS (ESI) for C₁₄H₁₆N₂O₂ calc. 244.12, found 245.03 [M+H]⁺.

2-(Tetrahydro-2H-pyran-2-yl)-3-(4,4,5,5-tetramethyl-1,3,2-dioxaborolan-2-yl)-4,5-dihydro-2H-furo[3,2-g]indazole. A mixture containing 2-(tetrahydro-2H-pyran-2-yl)-4,5-dihydro-2H-furo[3,2-g]indazole (0.19 g, 0.78 mmol) and anhydrous THF (4 mL) was cooled to −60° C. n-BuLi (0.37 mL, 0.93 mmol) was added dropwise and the reaction mixture was stirred for 5 minutes, then quenched with i-PrOBPin (0.19 mL, 0.93 mmol). The reaction mixture was allowed to warm gradually to room temperature, then diluted with water and extracted 3× with EtOAc. The combined organic layers were washed with brine and dried over sodium sulfate. The crude product was purified by silica gel chromatography in 10-20% EtOAc/hexanes, to afford 0.20 g (70% yield, 0.54 mmol) of 2-(tetrahydro-2H-pyran-2-yl)-3-(4,4,5,5-tetramethyl-1,3,2-dioxaborolan-2-yl)-4,5-dihydro-2H-furo[3,2-g]indazole as a viscous oil. ¹H NMR (600 MHz, CDCl₃): 7.34 (d, J=1.2 Hz, 1H), 6.30 (d, J=1.8 Hz, 1H), 5.77 (dd, J=10.2, 2.4 Hz, 1H), 4.01 (m, 1H), 3.63 (m, 1H), 2.95 (m, 2H), 2.72 (t, J=8.1 Hz, 2H), 2.56 (m, 1H), 2.05 (m, 1H), 1.97 (m, 1H), 1.67 (m, 2H), 1.51 (m, 1H), 1.32 (d, J=2.4 Hz, 12H). MS (ESI) for C₂₀H₂₇BN₂O₄ calc. 370.21, found 371.10 [M+H]⁺.

3-(1-((tert-Butyldimethylsilyl)oxy)naphthalen-2-yl)-2-(tetrahydro-2H-pyran-2-yl)-4,5-dihydro-2H-furo[3,2-g]indazole. A mixture containing 2-(tetrahydro-2H-pyran-2-yl)-3-(4,4,5,5-tetramethyl-1,3,2-dioxaborolan-2-yl)-4,5-dihydro-2H-furo[3,2-g]indazole (0.18 g, 0.49 mmol), ((2-bromonaphthalen-1-yl)oxy)(tert-butyl)dimethylsilane (0.11 g, 0.33 mmol), XPhos-Pd-G3 (14 mg, 0.02 mmol), XPhos (8 mg, 0.02 mmol), and K₃PO₄ (0.14 g, 0.66 mmol) in a mixture of dioxane (1.3 mL) and H₂O (0.3 mL) was degassed 3× with nitrogen and heated at 60° C. for 16 h. The reaction mixture was cooled to room temperature, loaded onto Celite, and purified by silica gel chromatography in 10-20% EtOAc/hexanes to provide 21 mg (13% yield, 0.04 mmol) of 3-(1-((tert-butyldimethylsilyl)oxy)naphthalen-2-yl)-2-(tetrahydro-2H-pyran-2-yl)-4,5-dihydro-2H-furo[3,2-g]indazole as a colorless, viscous oil. MS (ESI) for C₃₀H₃₆N₂O₃Si calc. 500.25, found 501.07 [M+H]⁺.

2-(4,5-Dihydro-2H-furo[3,2-g]indazol-3-yl)naphthalen-1-ol. A mixture containing 3-(1-((tert-butyldimethylsilypoxy)naphthalen-2-yl)-2-(tetrahydro-2H-pyran-2-yl)-4,5-dihydro-2H-furo[3,2-g]indazole (21 mg, 0.04 mmol) and MeOH (2 mL) was treated with 1 drop of conc. HCl. The reaction mixture was stirred at room temperature and monitored for completion by LCMS. Removal of the THP group was complete after 2 h, at which point a 1 M solution of TBAF in THF (2 mL) was added dropwise. The reaction was monitored by LCMS and, upon completion (0.5 h), quenched with 1 N HCl (3 mL) and loaded directly onto Celite. The product was purified by silica gel chromatography in 0-5% MeOH/DCM to provide 10 mg (83% yield, 0.03 mmol) of '1790 as a gray solid. ¹H NMR (600 MHz, CDCl₃): δ 11.60 (brs, 1H), 10.10 (brs 1H), 8.41 (dd, 1H, J=6.6 Hz, 3.6 Hz), 7.76 (dd, 1H, J=6.0 Hz, 3.6 Hz), 7.71 (d, 1H, J=8.4 Hz), 7.47 (dd, 2H, J=6.0 Hz, 3.0 Hz), 7.40-7.38 (m, 2H), 6.42 (d, 1H, J=1.8 Hz), 3.25 (t, 2H, J=8.4 Hz), 2.93 (t, 2H, J=8.4 Hz). ¹³C NMR (150 MHz, DMSO): δ 142.7, 134.1, 127.4, 126.9, 125.6, 125.4, 124.5, 123.4, 121.2, 118.6, 111.5, 110.8, 22.5, 21.7. MS (ESI) for C₁₉H₁₄N₂O₂ calc 302.11, found 302.91 [M+H]⁺.

### Example 40: Procedural Details of Synthesis of '0527: 2-(5-(furan-2-yl)-1,3,4-oxadiazol-2-yl)naphthalen-1-ol

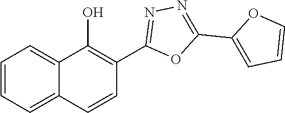 [SMILES: Oc1c(-c2nnc(-c3ccco3)o2)ccc2ccccc12]

(E)-N′-(Furan-2-ylmethylene)-1-hydroxy-2-naphthohydrazide: A solution containing 1-hydroxy-2-naphthohydrazide (0.30 g, 1.48 mmol) and furfural (0.13 mL, 1.56 mmol) in EtOH (7 mL) was heated at reflux for 2 h. The reaction mixture was allowed to cool to room temperature and the solvent was removed in vacuo. The crude product was dried in vacuo providing 0.396 g (1.41 mmol, 95% yield) of the (E)-N′-(furan-2-ylmethylene)-1-hydroxy-2-naphthohydrazide as a tan solid which was used with no further purification.

2-(5-(Furan-2-yl)-1,3,4-oxadiazol-2-yl)naphthalen-1-ol. (E)-N′-(furan-2-ylmethylene)-1-hydroxy-2-naphthohydrazide (0.270 g, 0.963 mmol) was suspended in anhydrous DCM (5 mL) and treated with [bis(trifluoroacetoxy)iodo]benzene (0.497 g, 1.16 mmol). The reaction mixture was stirred at room temperature for 16 h. The crude reaction mixture was loaded directly onto Celite and purified by column chromatography using 10-20% EtOAc/hexanes as an eluent. The off-white solids obtained were lixiviated with MeOH and dried in vacuo providing 0.068 g (0.244 mmol, 25% yield) of '0527 as an off-white solid. ¹H NMR (600 MHz, DMSO) δ 10.89 (s, 1H), 8.37 (dd, 1H, J=7.8, 0.6 Hz), 8.13 (dd, 1H, J=1.8, 0.6 Hz), 7.98 (d, 1H, J=8.4 Hz), 7.87 (d, 1H, J=8.4 Hz), 7.69 (dt, 1H, J=8.4, 1.8 Hz), 7.64 (dt, 2H, J=8.4, 1.2 Hz), 7.53 (dd, 1H, J=3.6, 0.6 Hz), 6.87 (dd, 1H, J=3.6, 1.8 Hz). ¹³C NMR (150 MHz, DMSO): δ 163.48, 155.69, 154.21, 147.31, 138.38, 135.55, 129.01, 127.90, 126.55, 124.06, 122.88, 122.45, 120.19, 115.27, 112.79, 101.78. MS (ESI) for C₁₆H₁₀N₂O₃ calc 278.07, found 278.77 [M+H]⁺.

### Example 41: Procedural Details of Synthesis of '9437: 2-(5-(tetrahydrofuran-2-yl)-1H-pyrazol-3-yl)naphthalen-1-ol

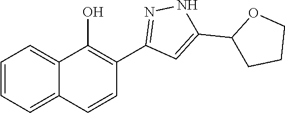 [SMILES: Oc1c(-c2cc(C3CCCO3)[nH]n2)ccc2ccccc12]

To a solution containing 3-(1-(benzyloxy)naphthalen-2-yl)-5-(furan-2-yl)-1H-pyrazole ('8882) (50 mg, 0.13 mmol). ethanol (15 mL), and ethyl acetate (15 mL) was added Pd/C (10%, 15 mg). The reaction mixture was allowed to stir 7 days in an atmosphere of H₂ (45 psi). Once judged complete by LCMS, the crude reaction was filtered over a celite plug. The plug was further washed with MeOH and the combined organic layers were evaporated under reduced pressure. The residue was purified by HPLC using 1% TFA (Aq)/MeCN as the eluent to afford '9437 (1.9 mg, 4.7%). LCMS (ESI) R_{T}=1.106 min, m/z=281.1 [M+H]⁺.

### Example 42: Procedural Details of Synthesis of '0457: 2-(5-(isopropylamino)-1H-pyrazol-3-yl)naphthalen-1-ol

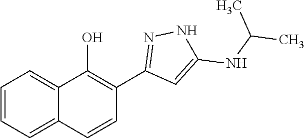 [SMILES: CC(C)Nc1cc(-c2ccc3ccccc3c2O)n[nH]1]

3-(1-Hydroxynaphthalen-2-yl)-3-oxopropanenitrile. To a solution containing iPr₂NH (330 μL, 2.37 mmol) and THF (10 mL) at −78° C. was added n-BuLi (1.6 M in THF, 1.4 mL, 2.25 mmol). The reaction mixture was allowed to warm to 0° C. over 30 min. MeCN (60 μL, 1.18 mmol) was then added and the reaction mixture was allowed to stir at 0° C. for an additional 2 h. At this time, a solution of methyl 1-hydroxy-2-naphthoate (150 mg, 0.74 mmol) in THF (4 mL) was added, and mixture was allowed to slowly warm to rt and stir for 16 h. The mixture was adsorbed on to celite and the crude product was purified by flash chromatography using EtOAc/Hexane (0-70%) as eluents to afford 3-(1-hydroxynaphthalen-2-yl)-3-oxopropanenitrile (108 mg, 69%). ¹H NMR (400 MHz, MeOD) δ (ppm) 8.51 (d, J=8.4 Hz, 1H), 7.81 (d, J=8.2 Hz, 1H), 7.74-7.69 (m, 1H), 7.63-7.58 (m, 1H), 7.44 (d, J=8.7 Hz, 1H), 7.34 (d, J=8.7 Hz, 1H), 4.22 (s, 2H). LCMS (ESI) R^{T}=0.959 min, m/z=214.2 [M+H]⁺

2-(5-Amino-1H-pyrazol-3-yl)naphthalen-1-ol. To a solution containing 3-(1-hydroxynaphthalen-2-yl)-3-oxopropanenitrile (100 mg, 0.47 mmol) and EtOH (10 mL) was added hydrazine (200 μL, 4.74 mmol). The reaction mixture was then heated at reflux for 16 h, then cooled to rt and adsorbed on to celite. The crude product was purified by flash chromatography using EtOAc/Hexane (0-70%) as eluents to afford 2-(5-amino-1H-pyrazol-3-yl)naphthalen-1-ol (79 mg, 74%). ¹H NMR (400 MHz, MeOD) δ (ppm) 8.33-8.27 (m, 1H), 7.88-7.84 (M, 1H), 7.65 (d, J=8.6 Hz, 1H), 7.58-7.53 (m, 2H), 7.51 (d, J=8.6 Hz, 1H). LCMS (ESI) R_{T}=0.776 min, m/z=226.3 [M+H]⁺

2-(5-(Isopropylamino)-1H-pyrazol-3-yl)naphthalen-1-ol. To a solution containing 4′(5-amino-1H-pyrazol-3-yl)naphthalen-1-ol (35 mg, 0.15 mmol) and DCM (15 mL) was added acetone (30 μL, 0.32 mmol), followed by sodium triacetoxyborohydride (130 mg, 0.32 mmol). The reaction mixture was allowed to stir at rt for 16 h. Once the starting material was consumed, MeOH (15 mL) was added and the mixture was allowed to stir for an additional 30 min. The crude product was then concentrated and purified by HPLC using 1% TFA (Aq)/MeCN as the eluent to afford 2-(5-(isopropylamino)-1H-pyrazol-3-yl)naphthalen-1-ol ('0457) (16.1 mg, 39%). ¹H NMR (MeOD) δ 8.32-8.21 (m, 1H), 7.87-7.80 (m, 2H), 7.66 (d, J=8.5 Hz, 1H), 7.89-7.42 (m 3H), 3.71-3.63 (m, 1H), 1.34-1.22 (m, 6H). LCMS (ESI) R_{T}=0.900 min, m/z=268.3 [M+H]⁺.

### Example 43: Human mAbs Against IsdA or IsdB Conjugated to Photosensitizers Mediate PDT-dependent Killing of S. aureus In Vitro

Human monoclonal antibodies were identified, which bind to iron-regulated IsdA or IsdB proteins on the surface of S. aureus(15, 18). Antibodies against these proteins protect against systemic murine infection. To expand the potential efficacy of these antibodies, a model of treatment was developed to use PDT, which is frequently used to treat bacterial skin infections(19). It involves the use of a photosensitizer and a light source to induce cell death through the production of reactive oxygen species (19). For these studies, the near-infrared photosensitizer IR700DX was conjugated to the Fc domains of IsdA or IsdB. This approach specifically delivers a photoactivating molecule proximal to the pathogen. Due to the specificity of the antibodies for the surface proteins of S. aureus, this method can selectively sensitize bacteria to light while avoiding host toxicity.

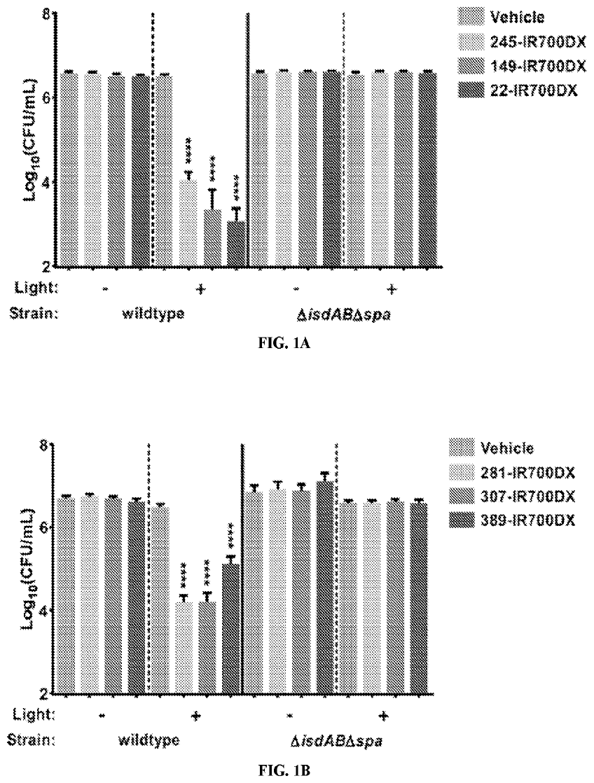

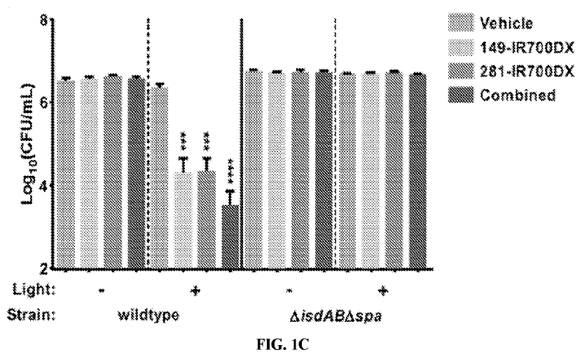

To determine the in vitro efficacy of PDT killing mediated through conjugated human mAbs targeting S. aureus Isd proteins, a light killing assay was designed. Wild-type Newman or a control strain lacking isdA, isdB, and protein A (ΔisdABΔspa) were iron-starved and then were exposed to αanti-IsdA or anti-IsdB antibodies conjugated with IR700DX prior to treatment with PDT light. Treatment with αanti-IsdA antibodies STAU-245, STAU-149, or STAU-22 or αanti-IsdB antibodies STAU-281, STAU-307, or STAU-389 conjugated with IR700DX inhibited growth of S. aureus following simultaneous exposure to 63 J/cm² of NIR (690 nm)-light and 50 J/cm² of blue (395 nm)-light, as compared to a vehicle-treated control (FIGS. 1A and 1B).

The ΔisdABΔspa strain did not exhibit changes in viability after treatment with any of the αanti-IsdA or αanti-IsdB antibodies followed by PDT. This finding was expected, as the relevant antigens are not available on the surface of that S. aureus strain. Each antibody was effective at inhibiting bacterial growth, but STAU-22 or STAU-149 were the most effective anti-IsdA antibodies, and STAU-281 or STAU-307 were the most effective anti-IsdB antibodies. Since the performance of STAU-22 could not be distinguished from that of STAU-149 and there was no discernable difference between STAU-281 and STAU-307 in individual antibody light killing comparisons, the previously published binding affinity of each of the antibodies was referenced to determine which antibody to advance from each group (15, 18).

An additive effect was seen when antibodies STAU-149 and STAU-281 were used in combination (FIG. 1C), due to simultaneous binding the antibodies to IsdA and IsdB on the surface of S. aureus and increased antibody occupancy. This is consistent with previously published data showing that combining conjugated antibodies decreased S. aureus viability by greater than 100-fold reduction in CFU compared with untreated cells (15). These results establish the utility of conjugated antibody-PDT as a potential therapeutic for the treatment of skin infections caused by S. aureus.

### Example 44: VU0038882 Analogs Upregulate S. aureus Heme Biosynthesis

Photosensitizer-conjugated antibodies expose bacteria to reactive oxygen species (ROS) externally, but this treatment can be paired with small molecule treatment to generate ROS internally via heme biosynthesis pathway intermediates. To discover new compounds capable of activating CgoX and/or displaying less toxicity to eukaryotic cells, a medicinal chemistry campaign was initatied based on systematic alternation of the '8882 structure. Each new analog was tested for the ability to activate HssRS using the previously described XylE colorimetric assay that employs a reporter construct in which the hrtAB promoter is fused to a xylE reporter gene. Compounds were tested to determine the magnitude by which they activated hrtAB gene expression in the absence of exogenously added heme(11, 25). This method allows for the detection of excess heme production, since basal synthesis of endogenous heme by S. aureus is not sufficient for activation of hrtAB(11).

TABLE 3
Compounds representative of 219 structural analogs to VU0038882.

| | | Specifc Activity @ 30 μM |
|---|---|---|
| Compound | Structure | (nMol/min*mg) |
| ′8882 | 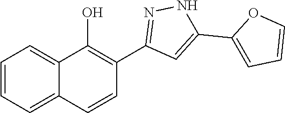 [SMILES: Oc1c(-c2cc(-c3ccco3)[nH]n2)ccc2ccccc12] | 30.04 ± 10.24 |
| ′2069 | 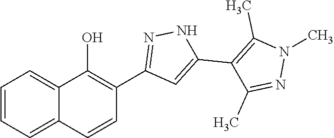 [SMILES: Cc1nn(C)c(C)c1-c1cc(-c2ccc3ccccc3c2O)n[nH]1] | 10.46 ± 1.54 |
| ′4083 | 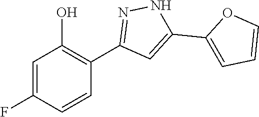 [SMILES: Oc1cc(F)ccc1-c1cc(-c2ccco2)[nH]n1] | 28.76 ± 9.41 |
| ′6700 | 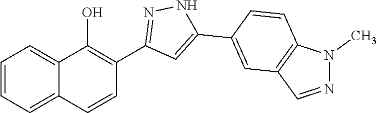 [SMILES: Cn1ncc2cc(-c3cc(-c4ccc5ccccc5c4O)n[nH]3)ccc21] | 24.26 ± 3.69 |
| ′9625 | 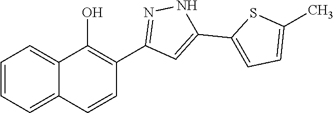 [SMILES: Cc1ccc(-c2cc(-c3ccc4ccccc4c3O)n[nH]2)s1] | 8.023 ± 1.065 |
| ′9626 | 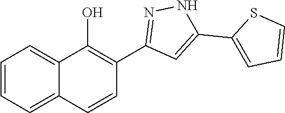 [SMILES: Oc1c(-c2cc(-c3cccs3)[nH]n2)ccc2ccccc12] | 28.86 ± 2.66 |
| ′9132 | 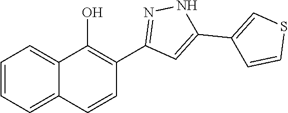 [SMILES: Oc1c(-c2cc(-c3ccsc3)[nH]n2)ccc2ccccc12] | 12.82 ± 1.12 |
| ′9133 | 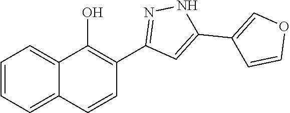 [SMILES: Oc1c(-c2cc(-c3ccoc3)[nH]n2)ccc2ccccc12] | 8.346 ± 1.304^{a} |
| ′9769 | 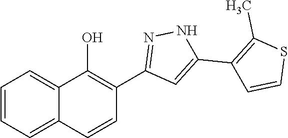 [SMILES: Cc1sccc1-c1cc(-c2ccc3ccccc3c2O)n[nH]1] | 4.922 ± 1.261 |
| ′9952 | 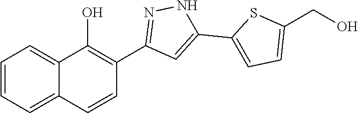 [SMILES: OCc1ccc(-c2cc(-c3ccc4ccccc4c3O)n[nH]2)s1] | 18.10 ± 1.80^{a} |
| ′0485 | 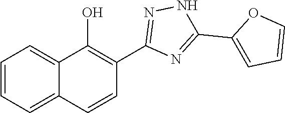 [SMILES: Oc1c(-c2n[nH]c(-c3ccco3)n2)ccc2ccccc12] | 1.697 ± 0.335 |
| 0394 | 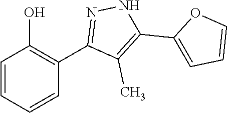 [SMILES: Cc1c(-c2ccccc2O)n[nH]c1-c1ccco1] | 0.0287 ± 0.0198 |
| ′1790 | 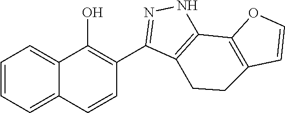 [SMILES: Oc1c(-c2n[nH]c3c2CCc2ccoc2-3)ccc2ccccc12] | 4.146 ± 2.397 |
| ′0527 | 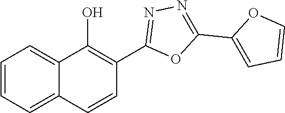 [SMILES: Oc1c(-c2nnc(-c3ccco3)o2)ccc2ccccc12] | 0.2270 ± 0.0147 |
| ′9437 | 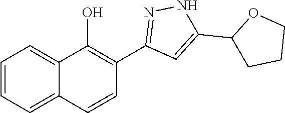 [SMILES: Oc1c(-c2cc(C3CCCO3)[nH]n2)ccc2ccccc12] | 0.088 ± 0.041 |
| '9624 | 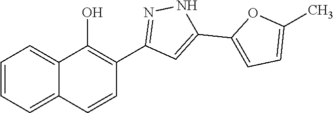 [SMILES: Cc1ccc(-c2cc(-c3ccc4ccccc4c3O)n[nH]2)o1] | 1.152 ± 0.382 |
| ′0071 | 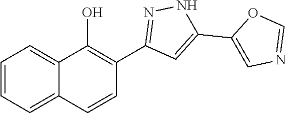 [SMILES: Oc1c(-c2cc(-c3cnco3)[nH]n2)ccc2ccccc12] | 1.152 ± 0.263^{a} |
| ′9134 | 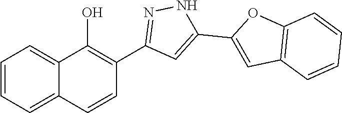 [SMILES: Oc1c(-c2cc(-c3cc4ccccc4o3)[nH]n2)ccc2ccccc12] | 0.130 ± 0.142 |
| ′9627 | 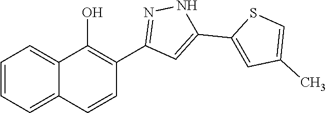 [SMILES: Cc1csc(-c2cc(-c3ccc4ccccc4c3O)n[nH]2)c1] | 10.15 ± 3.65 |
| ′9130 | 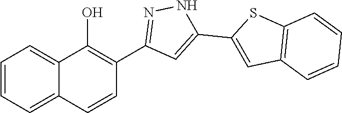 [SMILES: Oc1c(-c2cc(-c3cc4ccccc4s3)[nH]n2)ccc2ccccc12] | 1.012 ± 0.064 |
| ′8233 | 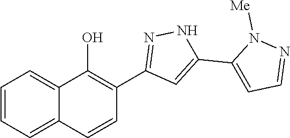 [SMILES: Cn1nccc1-c1cc(-c2ccc3ccccc3c2O)n[nH]1] | 0.5053 ± 0.294 |
| ′6699 | 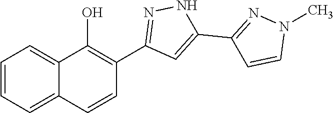 [SMILES: Cn1ccc(-c2cc(-c3ccc4ccccc4c3O)n[nH]2)n1] | 5.138 ± 4.82 |
| ′9131 | 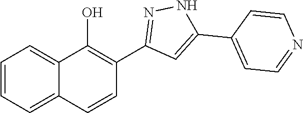 [SMILES: Oc1c(-c2cc(-c3ccncc3)[nH]n2)ccc2ccccc12] | 0.437 ± 0.26 |
| ′9767 | 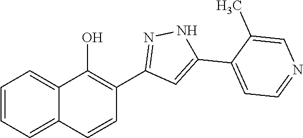 [SMILES: Cc1cnccc1-c1cc(-c2ccc3ccccc3c2O)n[nH]1] | 1.393 ± 0.392^{a} |
| ′9768 | 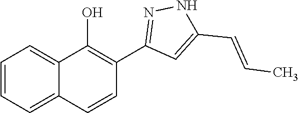 [SMILES: C/C=C/c1cc(-c2ccc3ccccc3c2O)n[nH]1] | 6.951 ± 0.983 |
| ′9128 | 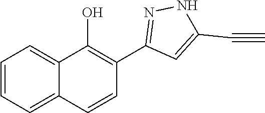 [SMILES: C#Cc1cc(-c2ccc3ccccc3c2O)n[nH]1] | 0.442 ± 0.089^{a} |
| ′0457 | 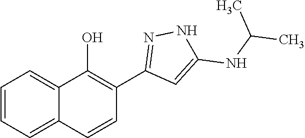 [SMILES: CC(C)Nc1cc(-c2ccc3ccccc3c2O)n[nH]1] | 0.5082 ± 0.770 |
| ′3918 | 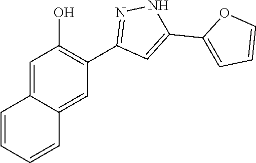 [SMILES: Oc1cc2ccccc2cc1-c1cc(-c2ccco2)[nH]n1] | 0.2543 ± 0.127 |
| ′4087 | 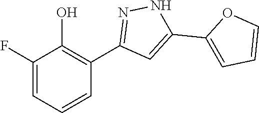 [SMILES: Oc1c(F)cccc1-c1cc(-c2ccco2)[nH]n1] | 5.119 ± 1.09 |
| ′4082 | 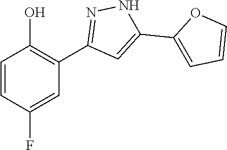 [SMILES: Oc1ccc(F)cc1-c1cc(-c2ccco2)[nH]n1] | 5.884 ± 1.88 |
| ′7268 | 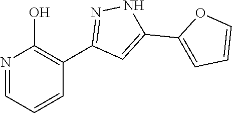 [SMILES: Oc1ncccc1-c1cc(-c2ccco2)[nH]n1] | 0.4793 ± 1.2179 |
| ′3989 | 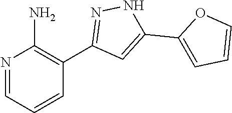 [SMILES: Nc1ncccc1-c1cc(-c2ccco2)[nH]n1] | 0.2491 ± 0.2546 |
| ′7092 | 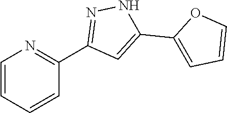 [SMILES: c1ccc(-c2cc(-c3ccco3)[nH]n2)nc1] | 0.2374 ± 0.1354 |

^{a}data collected at 10 uM

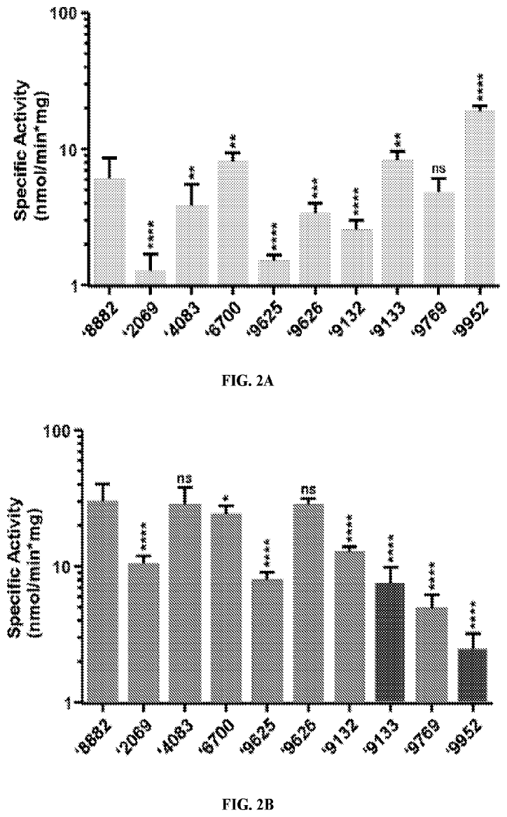

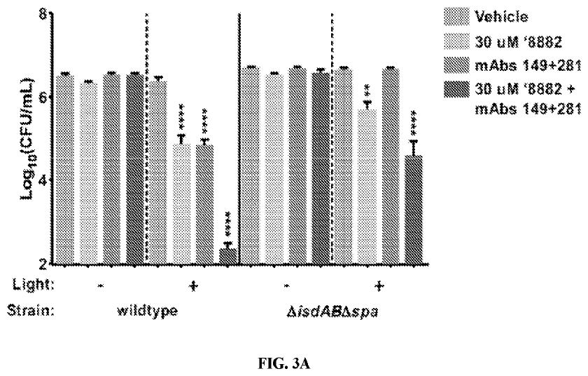

In total, 219 different '8882 analogs were prepared and tested (Examples 9-42). However, in general, only small changes to the molecular architecture allowed maintenance of activity (Table 3). For example, most changes to the naphthol ring, variations in the central pyrazole, and many furan alternatives led to significant diminishment of activity. Top performing analogs that led to notable HssRS-dependent upregulation of hrtAB expression after a six-hour exposure are shown in FIGS. 2A and 2B in comparison to '8882 at 10 μM (FIG. 2A) and 30 μM (FIG. 2B) in the XylE assay. These compounds comprise a fairly modest set of changes to the '8882 structure. For example, '9133, a positional furan isomer, outperformed '8882 at 10 μM.

Interestingly, '4083 features one of the few naphthol changes that maintains activity similar to '8882 at both doses. Some compositional changes to the furan ring were found to be tolerated. '2069 demonstrates weaker activity compared to '8882 at 10 μM, but is equivalent at 30 μM. Further, the benzopyrazole '6700 was among the analogs found to be roughly equivalent to '8882. The most consistent activity with non-furan-containing compounds was found with thiophene replacements. The direct thiophene analog '9626 performed well at 10 μM and was comparable to '8882 at 30 μM. Like with the furan, the positional thiophene isomer '9132 lost some activity compared with '8882. Substituting the thiophene with a hydroxymethyl resulted in '9952, with the highest activity at 10 μM observed to date. However, this compound, as well as the very active '9133, was found to be overtly toxic to bacteria at the target dose of 30 μM, suggesting that these compounds may act on targets other than CgoX. Since none of the compounds significantly outperformed '8882 in the ability to activate HssRS, and none were significantly less toxic to eukaryotic HepG2 cells, '8882 was selected as the primary small molecule advanced in these studies.

### Example 45: Simultaneous Exposure to IR700DX Conjugated Antibodies and '8882 Decreases S. aureus Viability Upon Light Exposure

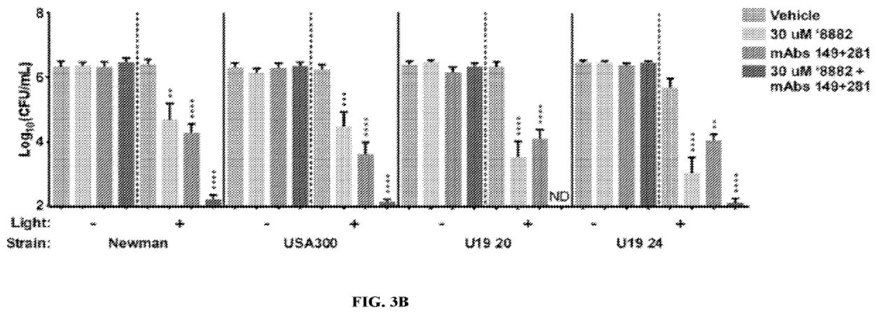

The data presented above indicate that the intracellular approach involving small molecule '8882 as well as the extracellular IR700DX-conjugated human mAbs alone are efficacious against S. aureus when used in PDT. It was contemplated that the combination of these treatments would have an enhanced effect on bacterial growth in vitro. A similar experimental design was maintained to directly compare the individual antibody and small molecule PDT treatments with the combinatorial strategy. Treatment with a mixture of both IsdA and IsdB antibodies conjugated with IR700DX along with small molecule '8882, led to significant growth inhibition of S. aureus following simultaneous exposure to 63 J/cm² of NIR (690 nm)-light and 50 J/cm² of blue (395 nm)-light (FIG. 3A). The enhanced effect observed is likely due to an increase in ROS present both internally and externally. Notably, the combined treatment decreased S. aureus viability by 10,000-fold compared with untreated cells. These experiments show the efficacy of antibody and small molecule mediated PDT against the methicillin-susceptible S. aureus (MSSA) strain Newman.

It was further contemplated that PDT treatment would be equally effective against MRSA strains, since MSSA and MRSA share the same proteins and enzymes used to maintain iron and heme homeostasis. Indeed, the clinically relevant MRSA strain USA300 NRS384 showed similar levels of growth inhibition upon PDT treatment (FIG. 3B). In addition, the recent clinically isolated MSSA strains U19 20 and U19 24 were as susceptible to PDT treatment as both Newman and USA300 NRS384 (FIG. 3B). These results establish the utility of combinatorial small molecule and conjugated antibody-PDT as a potential therapeutic for the treatment of skin infections caused by MRSA. Therefore, wild-type USA300 NRS384 was used in all assays described below to ensure clinical relevance.

### Example 46: STAU-149 (IsdA-specific) and STAU-281 (IsdB-specific) Bind to the Surface of Iron-starved S. aureus

The anti-IsdAB antibodies conjugated with IR700DX can be visualized with fluorescence microscopy, therefore it was contemplated that these antibodies could be used for diagnostic purposes in addition to therapeutic use. IR700DX is a photosensitizer that has an excitation wavelength of 689 nm and an emission wavelength of 700 nm, meaning it is visible in the NIR spectrum using fluorescence microscopy. This property is advantageous, since it is relatively easy to visualize labeled bacteria and the exposure should cause minimal absorption by tissue, including during infection.

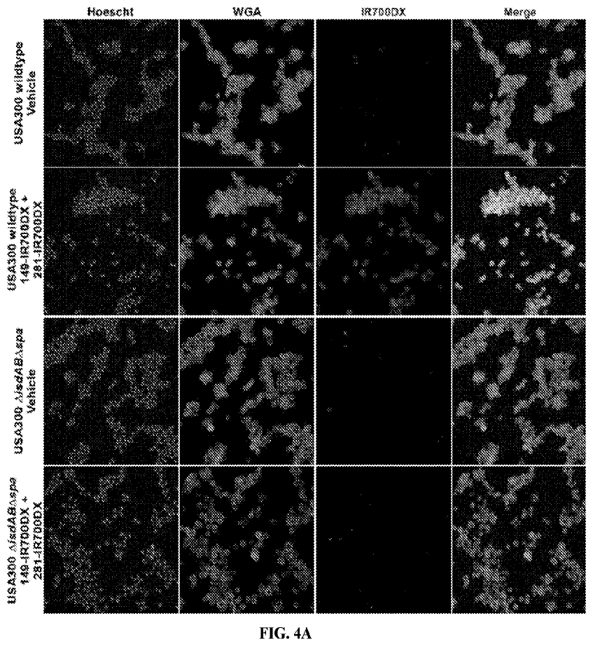

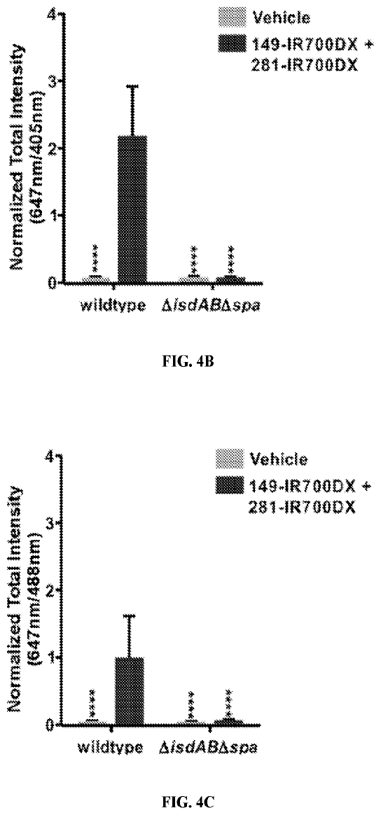

As expected, the fluorescence signal was evident when antibodies were applied to wild-type S. aureus, while signal was not detected when antibodies were applied to the control ΔisdABΔspa strain (FIG. 4A), indicating that the antibodies bind specifically to their target antigens. When comparing the total intensity of IR700DX signal, normalized to either the DNA signal from the Hoechst stain (FIG. 4B) or the cell wall WGA stain (FIG. 4C), there is significantly more signal in the wild-type strain exposed to antibodies as compared to any other condition. These results demonstrate that these antibodies bind specifically to their iron-regulated surface determinant antigens and represent potential diagnostic tools.

### Example 47: Combinatorial Intracellular and Extracellular PDT Decreases S. aureus Burdens In Vivo

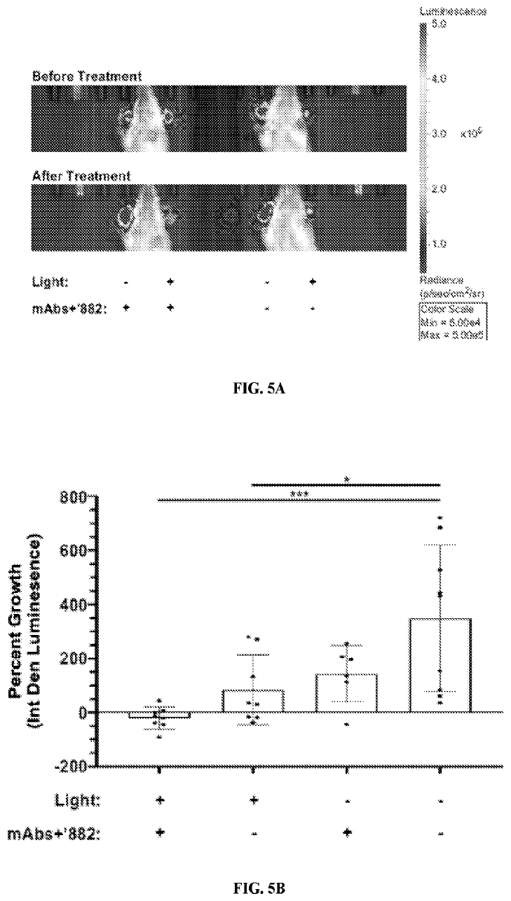

The results above demonstrate the efficacy of antibody and small-molecule-mediated antimicrobial PDT, the benefit of a combinatorial approach in therapeutic design, and the potential utility of these anti-Isd antibodies for diagnostic use in vitro. To determine the in vivo efficacy of antibody and '8882 combinatorial PDT, a murine model of superficial S. aureus skin infection was developed. Mice were pretreated intraperitoneally with 4 μg of antibodies, and 24 hours later each ear was tape-stripped to induce a superficial wound on the ventrodorsal surface of the ear. The wound was inoculated with 8×10⁶ CFUs of USA300 S. aureus constitutively expressing the luxBADCE cassette. After 240 minutes, 0.4 μg of antibody and 30 μM of '8882 were applied to the wound, and the mice were again injected intraperitoneally with antibodies. Each mouse was exposed to 690 nm and 395 nm light. After 24 hours, 0.4 μg of antibody was applied again, and then the mice were exposed to 690 nm light prior to the conclusion of the experiment. S. aureus burdens were tracked using bioluminescence imaging of the luciferase reporter before infection and at the conclusion of the experiment following treatment. Combined antibody- and '8882-mediated PDT led to a significant decrease in bacteria located on and around the wound compared with untreated mice (FIG. 5A). Mouse ears inoculated with the luminescent strain of S. aureus USA300 and then treated with '8882 and antibody-PDT exhibited significantly less bacterial growth measured by luminescence compared to untreated animals (FIGS. 5A and 5B). Reduction in burdens required combination treatment with the conjugated antibodies, '8882, and light. Mice treated with light alone or antibodies plus '8882 without light showed a reduction in bacterial burden but did not reach the same extent as the combinatorial treatment (FIG. 5B). These results highlight the utility of antibodies in combination with small molecules for PDT-dependent treatment of S. aureus infections in vivo. In addition, these findings establish an effective therapeutic strategy to target skin infections.

### Example 48: Discussion of Foregoing Examples

The public health threat of S. aureus necessitates the development of novel therapeutic strategies. Here, the value of a combinatorial PDT treatment against drug-resistant S. aureus exposed to a CgoX activator and conjugated anti-Isd mAbs is demonstrated. The specificity of these photoactivator-conjugated antibodies establishes these reagents as useful for not only PDT but also as biorecognition elements that could be incorporated into diagnostic tools. These results support the use of this treatment in a clinical context, so the clinically derived strain USA300 NRS384 with a luminescent modification was used in a murine ear PDT assay to show a significant reduction in bacterial burden. These results define the S. aureus-specific human mAbs and small molecule, '8882, in combination with aPDT as a potentially beneficial treatment for S. aureus skin infections.

The clinical relevance of PDT has risen to prominence as a valuable tool in the treatment of both solid tumor and metastatic cancer (26). The model for aPDT is directly inspired by the methods used in cancer PDT in that it combines the application of light with a photosensitizing agent to generate ROS leading to targeted cell death (27). Cancer PDT also uses a variety of photosensitizing agents including both porphyrins generated by the target cells and photosensitive dyes conjugated to antibodies (28, 29). Antibodies in cancer PDT are often conjugated with phthalocyanine dyes like IR700DX, since their absorption wavelength is near infrared and passes much more easily through tissue, which is paramount for deep tissue PDT (29, 30). Antibodies provide a particularly good microbial recognition tool in aPDT due to their target specificity and relatively long half-life when used in therapeutic doses (30, 31). Human mAbs have been previously shown to target the iron-regulated surface determinant system in vivo as the surface-exposed proteins are expressed under the iron-depleted conditions encountered in SSTIs (15, 18). These antibodies have now been shown to have aPDT-mediated therapeutic efficacy. In addition, these antibodies have the potential to be used as diagnostic tools for SSTIs due to their target specificity and near infrared detection wavelength.

A primary benefit of small molecules for PDT is the ability to enter a cell. The advantage of having small molecules that upregulate the heme biosynthesis pathway is twofold: the heme biosynthesis pathway produces photoactive intermediates, and the terminal steps in Gram-positive heme synthesis are unique and therefore represent candidate therapeutic targets. Therefore, small molecules that target the production of coproporphyrin III can be used for Gram-positive specific antimicrobial therapy. The application of '8882 in vitro was previously shown to have antimicrobial effects during PDT alone or in combination with the nonspecific treatment compound ALA (25). '8882 is disclosed herein to have a significant combinatorial effect with the antibodies described above during PDT. Analogs of '8882 were generated to explore the possibility of a more potent or more efficient small molecule. While a few analogs performed as well as or better than '8882 in the XylE assay, those results did not result in compounds with increased efficacy in the PDT.

The emergence of aPDT is progressing at a rapid rate as the scientific community is challenged by the evolution of antibiotic resistance in pathogenic bacteria. The primary variable studied in the PDT field is the photosensitizer, with emphasis on how well it can be taken up by bacteria and how well it produces ROS. There are four classes of commonly studied photosensitizers including natural photosensitizers, tetrapyrrole structures, phenothiazinium, and nano-structures. These classes have a wide variety of excitation wavelengths and levels of effectiveness, with notable examples being curcumin, methylene blue, and porphyrins from the natural, phenothiazinium, and tetrapyrrole structure classes respectively (32). Curcumin has been used in blue-light aPDT against notable bacterial species such as S. aureus, Bacillus subtilis, and Streptococcus pyogenes, and it has some anti-inflammatory side effects making it advantageous in the context of an infection (32, 33). Methylene blue on its own has low efficacy but has been used recently in conjugations with nanoparticles to inactivate species like S. aureus, Pseudomonas aeruginosa, and Escherichia coli (32, 34, 35). Porphyrins, such as CPIII, are useful because they are intermediates in heme biosynthesis, which has a conserved pathway specific to Gram-positive bacteria (9, 22, 23). Each of these photosensitizers are effective independently, but have drawbacks in either activation wavelength, uptake, or species specificity, which means a combinatorial strategy likely would be best for optimal aPDT.

The development of effective diagnostics is critical to the treatment of infectious diseases. Optical imaging is one of the most common methods by which infections are visualized due to the relative simplicity of acquiring images using these methods (36). Fluorescence imaging typically uses a probe with excitation and emission wavelengths designed to specifically target bacteria in the context of an infection. Some bacterial probes take advantage of anionic surface of bacterial cells by having complexes containing an affinity group on one end and a near-infrared fluorophore on the other (36, 37). These near-infrared probes are advantageous for internal or deep tissue infections since the near-infrared wavelengths will have minimal absorption passing through tissue. For SSTIs, there are benefits to having greater levels of specificity since these probes can also bind to apoptotic eukaryotic cells, which are much more common on the surface of the skin. Therefore, the photosensitizer-conjugated antibodies used in this study have particular utility in the optical imaging of Gram-positive bacteria in the context of SSTIs.

The combination of the small molecule and photosensitizer-conjugated anti-Isd human monoclonal antibodies was shown to have significant efficacy in vitro, but it was necessary to determine whether there was any biological relevance for its use in a therapeutic regimen. The significant difference in bacterial luminescent signal between the experimental and control conditions in the murine model gives evidence to support the idea that these antibodies and small molecules can be used to great effect in combination with photodynamic therapy. There is a nonsignificant difference between two of the control conditions: light alone and photosensitizers alone, which is a common phenotype in an in vivo model. This is likely due to the application of light alone accelerating wound healing and increasing the host immune response resulting in a minor decrease in bacterial burdens (25, 38, 39). There is great potential to further refine this animal model and to explore the use of this therapeutic regimen for other Gram-positive pathogen derived SSTIs. The evidence presented lends support to the conclusion that antimicrobial photodynamic therapy using small molecules and photosensitizer-conjugated antibodies can serve as an effective alternative or supplement to antibiotics.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Tognetti L, Martinelli C, Berti S, Hercogova J, Lotti T, Leoncini F, Moretti S. 2012. Bacterial skin and soft tissue infections: review of the epidemiology, microbiology, aetiopathogenesis and treatment: a collaboration between dermatologists and infectivologists. J Eur Acad Dermatol Venereol 26:931-41.
2. Anonymous. 2019. Antibiotic resistance threats in the United States, 2019.
3. Kourtis A P, Hatfield K, Baggs J, Mu Y, See I, Epson E, Nadle J, Kainer M A, Dumyati G, Petit S, Ray S M, Emerging Infections Program Mag, Ham D, Capers C, Ewing H, Coffin N, McDonald L C, Jernigan J, Cardo D. 2019. Vital Signs: Epidemiology and Recent Trends in Methicillin-Resistant and in Methicillin-Susceptible Staphylococcus aureus Bloodstream Infections—United States. MMWR Morb Mortal Wkly Rep 68:214-219.
4. Skaar E P, Gaspar A H, Schneewind O. 2004. IsdG and IsdI, heme-degrading enzymes in the cytoplasm of Staphylococcus aureus. J Biol Chem 279:436-43.
5. Wu R, Skaar E P, Zhang R, Joachimiak G, Gornicki P, Schneewind O, Joachimiak A. 2005. Staphylococcus aureus IsdG and IsdI, heme-degrading enzymes with structural similarity to monooxygenases. J Biol Chem 280:2840-6.
6. Milani M, Pesce A, Nardini M, Ouellet H, Ouellet Y, Dewilde S, Bocedi A, Ascenzi P, Guertin M, Moens L, Friedman J M, Wittenberg J B, Bolognesi M. 2005. Structural bases for heme binding and diatomic ligand recognition in truncated hemoglobins. J Inorg Biochem 99:97-109.
7. Poulos T L. 2014. Heme enzyme structure and function. Chem Rev 114:3919-62.
8. Dailey H A, Gerdes S, Dailey T A, Burch J S, Phillips J D. 2015. Noncanonical coproporphyrin-dependent bacterial heme biosynthesis pathway that does not use protoporphyrin. Proc Natl Acad Sci USA 112:2210-5.
9. Dailey H A, Dailey T A, Gerdes S, Jahn D, Jahn M, O'Brian M R, Warren M J. 2017. Prokaryotic Heme Biosynthesis: Multiple Pathways to a Common Essential Product. Microbiology and Molecular Biology Reviews 81:e00048-16.
10. Mike L A, Duffer B F, Stauff D L, Moore J L, Vitko N P, Aranmolate O, Kehl-Fie T E, Sullivan S, Reid P R, DuBois J L, Richardson A R, Caprioli R M, Sulikowski G A, Skaar E P. 2013. Activation of heme biosynthesis by a small molecule that is toxic to fermenting Staphylococcus aureus. Proc Natl Acad Sci USA 110:8206-11.
11. Torres V J, Stauff D L, Pishchany G, Bezbradica J S, Gordy L E, Iturregui J, Anderson K L, Dunman P M, Joyce S, Skaar E P. 2007. A Staphylococcus aureus regulatory system that responds to host heme and modulates virulence. Cell Host Microbe 1:109-19.
12. Stauff D L, Tones V J, Skaar E P. 2007. Signaling and DNA-binding activities of the Staphylococcus aureus HssR-HssS two-component system required for heme sensing. J Biol Chem 282:26111-21.
13. Choby J E, Skaar E P. 2016. Heme Synthesis and Acquisition in Bacterial Pathogens. J Mol Biol 428:3408-28.
14. Muryoi N, Tiedemann M T, Pluym M, Cheung J, Heinrichs D E, Stillman M J. 2008. Demonstration of the Iron-regulated Surface Determinant (Isd) Heme Transfer Pathway in Staphylococcus aureus. Journal of Biological Chemistry 283:28125-28136.
15. Bennett M R, Bombardi R G, Kose N, Parrish E H, Nagel M B, Petit R A, Read T D, Schey K L, Thomsen I P, Skaar E P, Crowe J E. 2019. Human mAbs to Staphylococcus aureus IsdA Provide Protection Through Both Heme-Blocking and Fc-Mediated Mechanisms. J Infect Dis 219:1264-1273.
16. Mazmanian S K, Ton-That H, Su K, Schneewind O. 2002. An iron-regulated sortase anchors a class of surface protein during Staphylococcus aureus pathogenesis. Proc Natl Acad Sci USA 99:2293-8.
17. Skaar E P, Schneewind O. 2004. Iron-regulated surface determinants (Isd) of Staphylococcus aureus: stealing iron from heme. Microbes Infect 6:390-7.
18. Bennett M R, Dong J, Bombardi R G, Soto C, Parrington H M, Nargi R S, Schoeder C T, Nagel M B, Schey K L, Meiler J, Skaar E P, Crowe J E. 2019. Human VH1-69 Gene-Encoded Human Monoclonal Antibodies against Staphylococcus aureus IsdB Use at Least Three Distinct Modes of Binding To Inhibit Bacterial Growth and Pathogenesis. mBio 10.
19. Wan M T, Lin J Y. 2014. Current evidence and applications of photodynamic therapy in dermatology. Clin Cosmet Investig Dermatol 7:145-63.
20. Wachowska M, Muchowicz A, Firczuk M, Gabrysiak M, Winiarska M, Wańczyk M, Bojarczuk K, Golab J. 2011. Aminolevulinic Acid (ALA) as a Prodrug in Photodynamic Therapy of Cancer. 16:4140-4164.
21. Walter A B, Simpson J, Jenkins J L, Skaar E P, Jansen E D. 2020. Optimization of optical parameters for improved photodynamic therapy of Staphylococcus aureus using endogenous coproporphyrin III. Photodiagnosis Photodyn Ther 29:101624.
22. Maisch T, Hackbarth S, Regensburger J, Felgentrager A, Baumler W, Landthaler M, Roder B. 2011. Photodynamic inactivation of multi-resistant bacteria (PIB)—a new approach to treat superficial infections in the 21st century. J Dtsch Dermatol Ges 9:360-6.
23. Morimoto K, Ozawa T, Awazu K, Ito N, Honda N, Matsumoto S, Tsuruta D. 2014. Photodynamic therapy using systemic administration of 5-aminolevulinic acid and a 410-nm wavelength light-emitting diode for methicillin-resistant Staphylococcus aureus-infected ulcers in mice. PLoS One 9:e105173.
24. Calzavara-Pinton P G, Venturini M, Sala R. 2005. A comprehensive overview of photodynamic therapy in the treatment of superficial fungal infections of the skin. J Photochem Photobiol B 78:1-6.
25. Surdel M C, Horvath D J, Jr., Lojek L J, Fullen A R, Simpson J, Duffer B F, Salleng K J, Ford J B, Jenkins J L, Nagarajan R, Teixeira PL, Albertolle M, Georgiev I S, Jansen E D, Sulikowski G A, Lacy D B, Dailey H A, Skaar E P. 2017. Antibacterial photosensitization through activation of coproporphyrinogen oxidase. Proc Natl Acad Sci USA 114:E6652-E6659.
26. Dabrowski J M, Arnaut L G. 2015. Photodynamic therapy (PDT) of cancer: from local to systemic treatment. Photochemical & Photobiological Sciences 14:1765-1780.
27. Allison R, Moghissi K, Downie G, Dixon K. 2011. Photodynamic therapy (PDT) for lung cancer. Photodiagnosis and Photodynamic Therapy 8:231-239.
28. Allison R R, Downie G H, Cuenca R, Hu X-H, Childs C J, Sibata C H. 2004. Photosensitizers in clinical PDT. Photodiagnosis and Photodynamic Therapy 1:27-42.
29. Sato K, Nagaya T, Choyke P L, Kobayashi H. 2015. Near Infrared Photoimmunotherapy in the Treatment of Pleural Disseminated NSCLC: Preclinical Experience. Theranostics 5:698-709.
30. Lum Y-L, Ng D K P, Luk J M, Fong W-P. Development of anti-cadherin-17 antibody-IR700 conjugate for photodynamic therapy against gastrointestinal cancers, p. In (ed), SPIE,
31. Ryman J T, Meibohm B. 2017. Pharmacokinetics of Monoclonal Antibodies. CPT: Pharmacometrics & Systems Pharmacology 6:576-588.
32. Ghorbani J, Rahban D, Aghamiri S, Teymouri A, Bahador A. 2018. Photosensitizers in antibacterial photodynamic therapy: an overview. LASER THERAPY 27:293-302.
33. Soares J M, Silva K O O, Inada N M, Bagnato V S, Blanco K C. 2020. Optimization for microbial incorporation and efficiency of photodynamic therapy using variation on curcumin formulation. Photodiagnosis and Photodynamic Therapy 29:101652.
34. Parasuraman P, R. Y T, Shaji C, Sharan A, Bahkali A H, Al-Harthi H F, Syed A, Anju V T, Dyavaiah M, Siddhardha B. 2020. Biogenic Silver Nanoparticles Decorated with Methylene Blue Potentiated the Photodynamic Inactivation of Pseudomonas aeruginosa and Staphylococcus aureus. Pharmaceutics 12:709.
35. Toledo V H, Yoshimura T M, Pereira S T, Castro C E, Ferreira F F, Ribeiro M S, Haddad P S. 2020. Methylene blue-covered superparamagnetic iron oxide nanoparticles combined with red light as a novel platform to fight non-local bacterial infections: A proof of concept study against Escherichia coli. Journal of Photochemistry and Photobiology B: Biology 209:111956.
36. Leevy W M, Gammon S T, Jiang H, Johnson J R, Maxwell D J, Jackson E N, Marquez M, Piwnica-Worms D, Smith B D. 2006. Optical Imaging of Bacterial Infection in Living Mice Using a Fluorescent Near-Infrared Molecular Probe. Journal of the American Chemical Society 128:16476-16477.
37. White A G, Fu N, Leevy W M, Lee J-J, Blasco M A, Smith BD. 2010. Optical Imaging of Bacterial Infection in Living Mice Using Deep-Red Fluorescent Squaraine Rotaxane Probes. 21:1297-1304.
38. Rochkind S, Rousso M, Nissan M, Villarreal M, Barr-Nea L, Rees D G. 1989. Systemic effects of low-power laser irradiation on the peripheral and central nervous system, cutaneous wounds, and burns. Lasers Surg Med 9:174-82.
39. Whelan H T, Smits R L, Jr., Buchman E V, Whelan N T, Turner S G, Margolis D A, Cevenini V, Stinson H, Ignatius R, Martin T, Cwiklinski J, Philippi A F, Graf W R, Hodgson B, Gould L, Kane M, Chen G, Caviness J. 2001. Effect of NASA light-emitting diode irradiation on wound healing. J Clin Laser Med Surg 19:305-14.
40. Pishchany G, Dickey S E, Skaar E P. 2009. Subcellular localization of the Staphylococcus aureus heme iron transport components IsdA and IsdB. Infect Immun 77:2624-34.
41. Ferrari M, Fornasiero M C, Isetta A M. 1990. MTT colorimetric assay for testing macrophage cytotoxic activity in vitro. J Immunol Methods 131:165-72.
42. Strober W. 2015. Trypan Blue Exclusion Test of Cell Viability. Curr Protoc Immunol 111:A3 B 1-A3 B 3.
43. Nichols D, Hicks R, Bhattacharjee A. 2004. Antimalarial and antiproliferative pharmacophore models, novel tryptanthrin compounds having increased solubility, and methods of making and using thereof patent US20040033934A1.
44. Kugelberg E, Norstrom T, Petersen T K, Duvold T, Andersson D I, Hughes D. 2005. Establishment of a superficial skin infection model in mice by using Staphylococcus aureus and Streptococcus pyogenes. Antimicrob Agents Chemother 49:3435-41.
45. Plaut R D, Mocca C P, Prabhakara R, Merkel T J, Stibitz S. 2013. Stably luminescent Staphylococcus aureus clinical strains for use in bioluminescent imaging. PLoS One 8:e59232.
46. Schneider C A, Rasband W S, Eliceiri K W. 2012. NIH Image to ImageJ: 25 years of image analysis. Nat Methods 9:671-5.
47. Rueden C T, Schindelin J, Hiner M C, DeZonia B E, Walter A E, Arena E T, Eliceiri K W. 2017. ImageJ2: ImageJ for the next generation of scientific image data. BMC Bioinformatics 18:529.
48. Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, Cardona A. 2012. Fiji: an open-source platform for biological-image analysis. Nat Methods 9:676-82.
49. Duthie E S, Lorenz L L. 1952. Staphylococcal coagulase; mode of action and antigenicity. J Gen Microbiol 6:95-107.
50. International Patent Application Publication No. WO 2020/060771 for Human Monoclonal Antibodies to Staphylococcal Aureaus Isd Proteins and Uses Thereof
51. Escobedo J O, Rusin O, Lim S, and Strongin R M. 2010. NIR Dyes for Bioimaging Applications. Curr Opin Chem 14 (1):64.
52. Zhu S, Tian R, Antaris A L, Chen X, and Dia H. 2019. Near-infrared-II molecular dyes for cancer imaging and surgery. Adv Mater 31 (24): e1900321

It will be understood that various details of the presently disclosed subject matter can be changed without departing from the scope of the subject matter disclosed herein. Furthermore, the foregoing description is for the purpose of illustration only, and not for the purpose of limitation.

1. A method of inducing death or inhibiting growth or reproduction of a Gram-positive bacterial cell, comprising:
(a) contacting the bacterial cell with a coproporphyrinogen oxidase (CgoX) activator, thereby promoting accumulation of coproporphyrin III (CPIII) in the bacterial cell;
(b) contacting the bacterial cell with a photosensitizer conjugated to an iron-regulated surface determinant (Isd) protein targeting moiety;
(c) exposing the bacterial cell to light having a first excitation wavelength of CPIII; and
(d) exposing the bacterial cell to light having a second excitation wavelength of the photosensitizer that is conjugated to the targeting moiety.

2. The method of claim 1, wherein the bacterial cell is Staphylococcus, Streptococcus, Enterococcus, Bacillus, Clostridium, or Listeria.

3. (canceled)

4. The method of claim 2, wherein the bacterial cell is S. aureus.

5. The method of claim 1, wherein the light of the first excitation wavelength is blue, violet or ultraviolet A (UVA).

6. The method of claim 1, wherein the first excitation wavelength is about 390 nm to about 420 nm.

7. The method of claim 1, wherein the photosensitizer is a red or near infrared (NIR) dye.

8. The method of claim 1, wherein the photosensitizer is a near infrared (NIR) dye.

9. The method of claim 1, wherein the photosensitizer is a dye having an excitation wavelength of about 680 nm to about 1400 nm.

10. The method of claim 1, wherein the photosensitizer is a dye having an excitation wavelength of about 700 nm to about 1200 nm.

11. The method of claim 1, wherein the photosensitizer is a dye having an excitation wavelength of about 650 nm to about 800 nm.

12. The method of claim 1, wherein the Isd targeting moiety is an IsdA targeting moiety or an IsdB targeting moiety.

13. The method of claim 1, wherein the Isd targeting moiety is an IsdA antibody or an IsdB antibody.

14. The method of claim 1, wherein the bacterial cell is in an animal.

15. The method of claim 14, wherein the bacterial cell is causing an infection.

16. The method of claim 15, wherein the infection is in skin or soft tissue of the animal.

17-18. (canceled)

19. A composition, comprising a combination of a CgX activator and a photosensitizer conjugated to an Isd-targeting moiety.

20. The composition of claim 19, and further comprising a pharmaceutically-acceptable carrier.

21. (canceled)

22. A method of treating a skin or soft tissue infection in an animal, comprising administering the composition of claim 19 to or adjacent to the infected skin or soft tissue, and exposing the skin or soft tissue to a first excitation wavelength of CPIII, and a second excitation wavelength of the photosensitizer.

23. A compound, selected from the group consisting of: '2069, '4083, '6700, '9625, '9132, '9133, '9769, '9952, '0485, '0394, '1790, '0527, '9437, '9624, '0071, '9134, '9627, '9130, '8233, '6699, '9131, '9767, '9768, '9128, '0457, '3918, '4087, '4082, '7268, '3989, and '7092.

24. The compound of claim 23, selected from the group consisting of: '9952, '9133, '6700, '9625, '9626, '4083, '9132, and '2069.
